SEQUENCE LISTING (64 sequences)

SEQ ID NO: 1 - protein, 217 aa, Artificial Sequence
  Synthetic Artificial Sequence-Amino Acid Sequence of Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 45 aa, Artificial Sequence
  Synthetic Artificial Sequence - Leucine Zipper c-JUN-His
RIARLEEKVKTLKAQNSELASTANMLREQVAQLKQKVMNHHHHHH

SEQ ID NO: 3 - protein, 45 aa, Artificial Sequence
  Synthetic Artificial Sequence- Leucine Zipper c-FOS-His
LTDTLQAETDQLEDEKSALQTEIANLLKEKEKLEFILAAHHHHHH

SEQ ID NO: 4 - protein, 214 aa, Artificial Sequence
  Synthetic Artificial Sequence- VL-CL Amino Acid Sequence of Anti-PD-L1 Fab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 221 aa, Artificial Sequence
  Synthetic Artificial Sequence- VH-CH1 Amino Acid Sequence of Anti-ii-PD-L1 Fab
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 25 aa, Artificial Sequence
  Synthetic Artificial Sequence-Amino Acid Sequence of Linker
GGGGSGGGGSEPKSCDKTHTCPPCP

SEQ ID NO: 7 - protein, 25 aa, Artificial Sequence
  Synthetic Artificial Sequence-Amino Acid Sequence of Linker1
DDDDKGSGSGEPKSCDKTHTCPPCP

SEQ ID NO: 8 - protein, 25 aa, Artificial Sequence
  Synthetic Artificial Sequence-Amino Acid Sequence of Linker2
GGGGSGGGGSEPKSSDKTHTCPPCP

SEQ ID NO: 9 - protein, 15 aa, Artificial Sequence
  Synthetic Artificial Sequence-Amino Acid Sequence of Linker3
GGGGSGGGGSGGGGS

SEQ ID NO: 10 - protein, 149 aa, Artificial Sequence
  Synthetic Artificial Sequence-Amino Acid Sequence of Wild Type LAG-3 Domain D1
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASM

SEQ ID NO: 11 - protein, 90 aa, Artificial Sequence
  Synthetic Artificial Sequence-Amino Acid Sequence of Wild Type LAG-3 Domain D2
TASPPGSLRASDWVILNCSFSRPDRPASVHWFRNRGQGRVPVRESPHHHLAESFLFLPQVSPMDSGPWGCILTYRDGFNVSIMYNLTVLG

SEQ ID NO: 12 - protein, 91 aa, Artificial Sequence
  Synthetic Artificial Sequence-Amino Acid Sequence of Wild Type LAG-3 Domain D3
LEPPTPLTVYAGAGSRVGLPCRLPAGVGTRSFLTAKWTPPGGGPDLLVTGDNGDFTLRLEDVSQAQAGTYTCHIHLQEQQLNATVTLAIIT

SEQ ID NO: 13 - protein, 82 aa, Artificial Sequence
  Synthetic Artificial Sequence-Amino Acid Sequence of Wild Type LAG-3 Domain D4
VTPKSFGSPGSLGKLLCEVTPVSGQERFVWSSLDTPSQRSFSGPWLEAQEAQLLSQPWQCQLYQGERLLGAAVYFTELSSPG

SEQ ID NO: 14 - protein, 90 aa, Artificial Sequence
  Synthetic Artificial Sequence-Amino Acid Sequence of D2 Domain Mutant D2-R188A
TASPPGSLRASDWVILNCSFSRPDRPASVHWFRNRGQGAVPVRESPHHHLAESFLFLPQVSPMDSGPWGCILTYRDGFNVSIMYNLTVLG

SEQ ID NO: 15 - protein, 90 aa, Artificial Sequence
  Synthetic Artificial Sequence-Amino Acid Sequence of D2 Domain Mutant D2-R192A
TASPPGSLRASDWVILNCSFSRPDRPASVHWFRNRGQGRVPVAESPHHHLAESFLFLPQVSPMDSGPWGCILTYRDGFNVSIMYNLTVLG

SEQ ID NO: 16 - protein, 90 aa, Artificial Sequence
  Synthetic Artificial Sequence-Amino Acid Sequence of D2 Domain Mutant D2-H196A
TASPPGSLRASDWVILNCSFSRPDRPASVHWFRNRGQGRVPVRESPAHHLAESFLFLPQVSPMDSGPWGCILTYRDGFNVSIMYNLTVLG

SEQ ID NO: 17 - protein, 90 aa, Artificial Sequence
  Synthetic Artificial Sequence-Amino Acid Sequence of D2 Domain Mutant D2-H197A
TASPPGSLRASDWVILNCSFSRPDRPASVHWFRNRGQGRVPVRESPHAHLAESFLFLPQVSPMDSGPWGCILTYRDGFNVSIMYNLTVLG

SEQ ID NO: 18 - protein, 90 aa, Artificial Sequence
  Synthetic Artificial Sequence-Amino Acid Sequence of D2 Domain Mutant D2-P175A
TASPPGSLRASDWVILNCSFSRPDRAASVHWFRNRGQGRVPVRESPHHHLAESFLFLPQVSPMDSGPWGCILTYRDGFNVSIMYNLTVLG

SEQ ID NO: 19 - protein, 90 aa, Artificial Sequence
  Synthetic Artificial Sequence-Amino Acid Sequence of D2 Domain Mutant D2-S177A
TASPPGSLRASDWVILNCSFSRPDRPAAVHWFRNRGQGRVPVRESPHHHLAESFLFLPQVSPMDSGPWGCILTYRDGFNVSIMYNLTVLG

SEQ ID NO: 20 - protein, 90 aa, Artificial Sequence
  Synthetic Artificial Sequence-Amino Acid Sequence of D2 Domain Mutant D2-V178A
TASPPGSLRASDWVILNCSFSRPDRPASAHWFRNRGQGRVPVRESPHHHLAESFLFLPQVSPMDSGPWGCILTYRDGFNVSIMYNLTVLG

SEQ ID NO: 21 - protein, 90 aa, Artificial Sequence
  Synthetic Artificial Sequence-Amino Acid Sequence of D2 Domain Mutant D2-N183A
TASPPGSLRASDWVILNCSFSRPDRPASVHWFRARGQGRVPVRESPHHHLAESFLFLPQVSPMDSGPWGCILTYRDGFNVSIMYNLTVLG

SEQ ID NO: 22 - protein, 90 aa, Artificial Sequence
  Synthetic Artificial Sequence-Amino Acid Sequence of D2 Domain Mutant D2-G185A
TASPPGSLRASDWVILNCSFSRPDRPASVHWFRNRAQGRVPVRESPHHHLAESFLFLPQVSPMDSGPWGCILTYRDGFNVSIMYNLTVLG

SEQ ID NO: 23 - protein, 90 aa, Artificial Sequence
  Synthetic Artificial Sequence-Amino Acid Sequence of D2 Domain Mutant D2-Q186A
TASPPGSLRASDWVILNCSFSRPDRPASVHWFRNRGAGRVPVRESPHHHLAESFLFLPQVSPMDSGPWGCILTYRDGFNVSIMYNLTVLG

SEQ ID NO: 24 - protein, 90 aa, Artificial Sequence
  Synthetic Artificial Sequence-Amino Acid Sequence of D2 Domain Mutant D2-G187A
TASPPGSLRASDWVILNCSFSRPDRPASVHWFRNRGQARVPVRESPHHHLAESFLFLPQVSPMDSGPWGCILTYRDGFNVSIMYNLTVLG

SEQ ID NO: 25 - protein, 90 aa, Artificial Sequence
  Synthetic Artificial Sequence-Amino Acid Sequence of D2 Domain Mutant D2-V189A
TASPPGSLRASDWVILNCSFSRPDRPASVHWFRNRGQGRAPVRESPHHHLAESFLFLPQVSPMDSGPWGCILTYRDGFNVSIMYNLTVLG

SEQ ID NO: 26 - protein, 90 aa, Artificial Sequence
  Synthetic Artificial Sequence-Amino Acid Sequence of D2 Domain Mutant D2-P190A
TASPPGSLRASDWVILNCSFSRPDRPASVHWFRNRGQGRVAVRESPHHHLAESFLFLPQVSPMDSGPWGCILTYRDGFNVSIMYNLTVLG

SEQ ID NO: 27 - protein, 90 aa, Artificial Sequence
  Synthetic Artificial Sequence-Amino Acid Sequence of D2 Domain Mutant D2-P195A
TASPPGSLRASDWVILNCSFSRPDRPASVHWFRNRGQGRVPVRESAHHHLAESFLFLPQVSPMDSGPWGCILTYRDGFNVSIMYNLTVLG

SEQ ID NO: 28 - protein, 90 aa, Artificial Sequence
  Synthetic Artificial Sequence-Amino Acid Sequence of D2 Domain Mutant D2-L199A
TASPPGSLRASDWVILNCSFSRPDRPASVHWFRNRGQGRVPVRESPHHHAAESFLFLPQVSPMDSGPWGCILTYRDGFNVSIMYNLTVLG

SEQ ID NO: 29 - protein, 90 aa, Artificial Sequence
  Synthetic Artificial Sequence-Amino Acid Sequence of D2 Domain Mutant D2-F203A
TASPPGSLRASDWVILNCSFSRPDRPASVHWFRNRGQGRVPVRESPHHHLAESALFLPQVSPMDSGPWGCILTYRDGFNVSIMYNLTVLG

SEQ ID NO: 30 - protein, 90 aa, Artificial Sequence
  Synthetic Artificial Sequence-Amino Acid Sequence of D2 Domain Mutant D2-Q208A
TASPPGSLRASDWVILNCSFSRPDRPASVHWFRNRGQGRVPVRESPHHHLAESFLFLPAVSPMDSGPWGCILTYRDGFNVSIMYNLTVLG

SEQ ID NO: 31 - protein, 90 aa, Artificial Sequence
  Synthetic Artificial Sequence-Amino Acid Sequence of D2 Domain Mutant D2-S210A
TASPPGSLRASDWVILNCSFSRPDRPASVHWFRNRGQGRVPVRESPHHHLAESFLFLPQVAPMDSGPWGCILTYRDGFNVSIMYNLTVLG

SEQ ID NO: 32 - protein, 90 aa, Artificial Sequence
  Synthetic Artificial Sequence-Amino Acid Sequence of D2 Domain Mutant D2-P211A
TASPPGSLRASDWVILNCSFSRPDRPASVHWFRNRGQGRVPVRESPHHHLAESFLFLPQVSAMDSGPWGCILTYRDGFNVSIMYNLTVLG

SEQ ID NO: 33 - protein, 90 aa, Artificial Sequence
  Synthetic Artificial Sequence-Amino Acid Sequence of D2 Domain Mutant D2-M212A
TASPPGSLRASDWVILNCSFSRPDRPASVHWFRNRGQGRVPVRESPHHHLAESFLFLPQVSPADSGPWGCILTYRDGFNVSIMYNLTVLG

SEQ ID NO: 34 - protein, 90 aa, Artificial Sequence
  Synthetic Artificial Sequence-Amino Acid Sequence of D2 Domain Mutant D2-S214A
TASPPGSLRASDWVILNCSFSRPDRPASVHWFRNRGQGRVPVRESPHHHLAESFLFLPQVSPMDAGPWGCILTYRDGFNVSIMYNLTVLG

SEQ ID NO: 35 - protein, 90 aa, Artificial Sequence
  Synthetic Artificial Sequence-Amino Acid Sequence of D2 Domain Mutant D2-P216A
TASPPGSLRASDWVILNCSFSRPDRPASVHWFRNRGQGRVPVRESPHHHLAESFLFLPQVSPMDSGAWGCILTYRDGFNVSIMYNLTVLG

SEQ ID NO: 36 - protein, 90 aa, Artificial Sequence
  Synthetic Artificial Sequence-Amino Acid Sequence of D2 Domain Mutant D2-G218A
TASPPGSLRASDWVILNCSFSRPDRPASVHWFRNRGQGRVPVRESPHHHLAESFLFLPQVSPMDSGPWACILTYRDGFNVSIMYNLTVLG

SEQ ID NO: 37 - protein, 90 aa, Artificial Sequence
  Synthetic Artificial Sequence-Amino Acid Sequence of D2 Domain Mutant D2-H198G
TASPPGSLRASDWVILNCSFSRPDRPASVHWFRNRGQGRVPVRESPHHGLAESFLFLPQVSPMDSGPWGCILTYRDGFNVSIMYNLTVLG

SEQ ID NO: 38 - protein, 90 aa, Artificial Sequence
  Synthetic Artificial Sequence-Amino Acid Sequence of D2 Domain Mutant D2-H198L
TASPPGSLRASDWVILNCSFSRPDRPASVHWFRNRGQGRVPVRESPHHLLAESFLFLPQVSPMDSGPWGCILTYRDGFNVSIMYNLTVLG

SEQ ID NO: 39 - protein, 90 aa, Artificial Sequence
  Synthetic Artificial Sequence-Amino Acid Sequence of D2 Domain Mutant D2-H198M
TASPPGSLRASDWVILNCSFSRPDRPASVHWFRNRGQGRVPVRESPHHMLAESFLFLPQVSPMDSGPWGCILTYRDGFNVSIMYNLTVLG

SEQ ID NO: 40 - protein, 90 aa, Artificial Sequence
  Synthetic Artificial Sequence-Amino Acid Sequence of D2 Domain Mutant D2-H198W
TASPPGSLRASDWVILNCSFSRPDRPASVHWFRNRGQGRVPVRESPHHWLAESFLFLPQVSPMDSGPWGCILTYRDGFNVSIMYNLTVLG

SEQ ID NO: 41 - protein, 90 aa, Artificial Sequence
  Synthetic Artificial Sequence-Amino Acid Sequence of D2 Domain Mutant D2-H198Y
TASPPGSLRASDWVILNCSFSRPDRPASVHWFRNRGQGRVPVRESPHHYLAESFLFLPQVSPMDSGPWGCILTYRDGFNVSIMYNLTVLG

SEQ ID NO: 42 - protein, 90 aa, Artificial Sequence
  Synthetic Artificial Sequence-Amino Acid Sequence of D2 Domain Mutant D2-H198V
TASPPGSLRASDWVILNCSFSRPDRPASVHWFRNRGQGRVPVRESPHHVLAESFLFLPQVSPMDSGPWGCILTYRDGFNVSIMYNLTVLG

SEQ ID NO: 43 - protein, 90 aa, Artificial Sequence
  Synthetic Artificial Sequence-Amino Acid Sequence of D2 Domain Mutant D2-E201R
TASPPGSLRASDWVILNCSFSRPDRPASVHWFRNRGQGRVPVRESPHHHLARSFLFLPQVSPMDSGPWGCILTYRDGFNVSIMYNLTVLG

SEQ ID NO: 44 - protein, 90 aa, Artificial Sequence
  Synthetic Artificial Sequence-Amino Acid Sequence of D2 Domain Mutant D2-E201N
TASPPGSLRASDWVILNCSFSRPDRPASVHWFRNRGQGRVPVRESPHHHLANSFLFLPQVSPMDSGPWGCILTYRDGFNVSIMYNLTVLG

SEQ ID NO: 45 - protein, 90 aa, Artificial Sequence
  Synthetic Artificial Sequence-Amino Acid Sequence of D2 Domain Mutant D2-E201D
TASPPGSLRASDWVILNCSFSRPDRPASVHWFRNRGQGRVPVRESPHHHLADSFLFLPQVSPMDSGPWGCILTYRDGFNVSIMYNLTVLG

SEQ ID NO: 46 - protein, 90 aa, Artificial Sequence
  Synthetic Artificial Sequence-Amino Acid Sequence of D2 Domain Mutant D2-E201Q
TASPPGSLRASDWVILNCSFSRPDRPASVHWFRNRGQGRVPVRESPHHHLAQSFLFLPQVSPMDSGPWGCILTYRDGFNVSIMYNLTVLG

SEQ ID NO: 47 - protein, 90 aa, Artificial Sequence
  Synthetic Artificial Sequence-Amino Acid Sequence of D2 Domain Mutant D2-E201H
TASPPGSLRASDWVILNCSFSRPDRPASVHWFRNRGQGRVPVRESPHHHLAHSFLFLPQVSPMDSGPWGCILTYRDGFNVSIMYNLTVLG

SEQ ID NO: 48 - protein, 90 aa, Artificial Sequence
  Synthetic Artificial Sequence-Amino Acid Sequence of D2 Domain Mutant D2-E201G
TASPPGSLRASDWVILNCSFSRPDRPASVHWFRNRGQGRVPVRESPHHHLAGSFLFLPQVSPMDSGPWGCILTYRDGFNVSIMYNLTVLG

SEQ ID NO: 49 - protein, 90 aa, Artificial Sequence
  Synthetic Artificial Sequence-Amino Acid Sequence of D2 Domain Mutant D2-E201F
TASPPGSLRASDWVILNCSFSRPDRPASVHWFRNRGQGRVPVRESPHHHLAFSFLFLPQVSPMDSGPWGCILTYRDGFNVSIMYNLTVLG

SEQ ID NO: 50 - protein, 90 aa, Artificial Sequence
  Synthetic Artificial Sequence-Amino Acid Sequence of D2 Domain Mutant D2-E201S
TASPPGSLRASDWVILNCSFSRPDRPASVHWFRNRGQGRVPVRESPHHHLASSFLFLPQVSPMDSGPWGCILTYRDGFNVSIMYNLTVLG

SEQ ID NO: 51 - protein, 90 aa, Artificial Sequence
  Synthetic Artificial Sequence-Amino Acid Sequence of D2 Domain Mutant D2-P207R
TASPPGSLRASDWVILNCSFSRPDRPASVHWFRNRGQGRVPVRESPHHHLAESFLFLRQVSPMDSGPWGCILTYRDGFNVSIMYNLTVLG

SEQ ID NO: 52 - protein, 90 aa, Artificial Sequence
  Synthetic Artificial Sequence-Amino Acid Sequence of D2 Domain Mutant D2-P207D
TASPPGSLRASDWVILNCSFSRPDRPASVHWFRNRGQGRVPVRESPHHHLAESFLFLDQVSPMDSGPWGCILTYRDGFNVSIMYNLTVLG

SEQ ID NO: 53 - protein, 90 aa, Artificial Sequence
  Synthetic Artificial Sequence-Amino Acid Sequence of D2 Domain Mutant D2-P207E
TASPPGSLRASDWVILNCSFSRPDRPASVHWFRNRGQGRVPVRESPHHHLAESFLFLEQVSPMDSGPWGCILTYRDGFNVSIMYNLTVLG

SEQ ID NO: 54 - protein, 90 aa, Artificial Sequence
  Synthetic Artificial Sequence-Amino Acid Sequence of D2 Domain Mutant D2-P207I
TASPPGSLRASDWVILNCSFSRPDRPASVHWFRNRGQGRVPVRESPHHHLAESFLFLIQVSPMDSGPWGCILTYRDGFNVSIMYNLTVLG

SEQ ID NO: 55 - protein, 90 aa, Artificial Sequence
  Synthetic Artificial Sequence-Amino Acid Sequence of D2 Domain Mutant D2-P207M
TASPPGSLRASDWVILNCSFSRPDRPASVHWFRNRGQGRVPVRESPHHHLAESFLFLMQVSPMDSGPWGCILTYRDGFNVSIMYNLTVLG

SEQ ID NO: 56 - protein, 90 aa, Artificial Sequence
  Synthetic Artificial Sequence-Amino Acid Sequence of D2 Domain Mutant D2-P207S
TASPPGSLRASDWVILNCSFSRPDRPASVHWFRNRGQGRVPVRESPHHHLAESFLFLSQVSPMDSGPWGCILTYRDGFNVSIMYNLTVLG

SEQ ID NO: 57 - protein, 90 aa, Artificial Sequence
  Synthetic Artificial Sequence-Amino Acid Sequence of D2 Domain Mutant D2-P207T
TASPPGSLRASDWVILNCSFSRPDRPASVHWFRNRGQGRVPVRESPHHHLAESFLFLTQVSPMDSGPWGCILTYRDGFNVSIMYNLTVLG

SEQ ID NO: 58 - protein, 90 aa, Artificial Sequence
  Synthetic Artificial Sequence-Amino Acid Sequence of D2 Domain Mutant D2-P207Y
TASPPGSLRASDWVILNCSFSRPDRPASVHWFRNRGQGRVPVRESPHHHLAESFLFLYQVSPMDSGPWGCILTYRDGFNVSIMYNLTVLG

SEQ ID NO: 59 - protein, 90 aa, Artificial Sequence
  Synthetic Artificial Sequence-Amino Acid Sequence of D2 Domain Mutant D2-V209T
TASPPGSLRASDWVILNCSFSRPDRPASVHWFRNRGQGRVPVRESPHHHLAESFLFLPQTSPMDSGPWGCILTYRDGFNVSIMYNLTVLG

SEQ ID NO: 60 - protein, 218 aa, Artificial Sequence
  Synthetic Artificial Sequence-VL-CL Amino Acid sequence of Anti-PD-1 Fab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 223 aa, Artificial Sequence
  Synthetic Artificial Sequence-VH-CH1 Amino Acid Sequence of Anti-PD-1 Fab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 62 - protein, 239 aa, Artificial Sequence
  Synthetic Artificial Sequence-Amino Acid Sequence of Wild Type LAG-3 D1-D2 Domain
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLG

SEQ ID NO: 63 - protein, 525 aa, Artificial Sequence
  Synthetic Artificial Sequence-Amino Acid Sequence of Human LAG-3 (including signal peptide)
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 64 - protein, 90 aa, Artificial Sequence
  Synthetic Artificial Sequence-Amino Acid Sequence of LAG-3 D2 domain mutant  P172A
TASPPGSLRASDWVILNCSFSRADRPASVHWFRNRGQGRVPVRESPHHHLAESFLFLPQVSPMDSGPWGCILTYRDGFNVSIMYNLTVLG

(19) United States
(12) Patent Application Publication
(10) Pub. No.: US 2024/0247043 A1
(43) Pub. Date: Jul. 25, 2024

(54) LAG-3 PROTEIN MUTANT, AND PREPARATION AND USE THEREOF

(71) Applicant: Wuhan YZY Biopharma Co., LTD., Wuhan (CN)

(72) Inventors: Lijuan Fang, Wuhan (CN); Jing Zhang, Wuhan (CN); Cheng Gong, Wuhan (CN); Xin Wang, Wuhan (CN); Fang Luo, Wuhan (CN); Yongxiang Yan, Wuhan (CN); Pengfei Zhou, Wuhan (CN)

(21) Appl. No.: 18/289,887

(22) PCT Filed: May 7, 2022
(86) PCT No.: PCT/CN2022/091464

§ 371 Date: Nov. 7, 2023

(30) Foreign Application Priority Data
May 8, 2021 (CN) 202110503020.0

(51) Int. Cl. C07K 14/705; A61K 38/17; A61K 45/06; A61K 47/64; A61K 49/00; A61K 51/08

(52) U.S. Cl. CPC C07K 14/70503; A61K 38/1774; A61K 45/06; A61K 47/6425; A61K 49/0056; A61K 51/08; C07K 2319/30

(57) ABSTRACT

The present invention relates to a LAG-3 protein mutant, and a fusion protein and the use thereof. The LAG-3 protein mutant of the present invention has mutations at one or more of the following positions in the domain 2 of a LAG-3 protein: 188, 192, 196, 197, 172, 175, 177, 178, 183, 185, 186, 187, 189, 190, 195, 199, 203, 208, 210, 211, 212, 214, 216, 218, 198, 201, 207 and 209.

### TECHNICAL FIELD

The present invention relates to the field of biopharmaceutics, and in particular, to an LAG-3 fusion protein mutant, a method for preparing same, and use thereof.

### BACKGROUND

Lymphocyte-activation gene 3 (LAG-3, or CD223) encodes a 498-amino acid type I transmembrane protein consisting of an extracellular region, a transmembrane region, and an intracellular region. The extracellular region has four Ig-like domains designated domain 1 to domain 4 (D1-D4). The extracellular region is similar to CD4, but has only 20% amino acid homology to CD4. The intracellular region consists of 3 parts: a serine phosphorylation site, a “KIEELE” motif, and EP repeats, wherein the “KIEELE” motif is a highly conserved sequence that is involved in intracellular signal transduction, but is not found in other proteins.

Under physiological conditions, LAG-3 is mainly expressed on the cell membrane of activated T cells, NK cells, B cells, and dendritic cells, and modulates the immune response of T cells by three predominant means: direct inhibition of T cell proliferation and activation through negative regulation; indirect inhibition of T cell responses by promoting inhibitory function of regulatory T cells (Treg); and prevention of T cell activation by modulating the function of antigen-presenting cells (APCs). LAG-3 inhibits T cell activation by transmitting inhibitory signals through the intracellular region.

LAG-3 distinguishes the conformation of pMHCII and selectively binds to stabilized pMHCII. To date, several molecules have been reported as potential ligands of LAG-3 in addition to the stabilized pMHCII. Galectin-3 and liver sinusoidal endothelial cell lectin (LSECtin) have been demonstrated to interact with the glycans on LAG-3. In 2019, Chen Lieping et al. demonstrated that FGL1 is an important functional ligand of LAG-3, and revealed that the LAG-3-FGL1 pathway is another tumor immune escape pathway independent of the PD-L1-PD-1 pathway, and the blockage of the pathway may play a synergistic role with anti-PD-1 therapies.

Current pharmaceutical development for LAG-3 includes anti-LAG-3 blocking antibodies, depleting antibodies, agonist antibodies, and fusion proteins of LAG-3. IMP321 is a soluble recombinant fusion protein consisting of the extracellular region of LAG-3 and the Fc domain of IgG, and can activate antigen-presenting cells by MHCII-mediated reverse signaling, resulting in increased IL-12 and TNF and the up-regulation of CD80 and CD86. Use of the drug for cancer treatment is currently in the clinical stage.

### SUMMARY

The present invention relates to an LAG-3 protein mutant, a fusion protein thereof, and use thereof. Specifically, the present invention relates to the following: 1. An LAG-3 protein mutant, comprising mutations at one or more of the following positions on the basis of the domain 2 of the LAG-3 protein: 188, 192, 196, 197, 172, 175, 177, 178, 183, 185, 186, 187, 189, 190, 195, 199, 203, 208, 210, 211, 212, 214, 216, 218, 198, 201, 207 and 209, preferably, mutations at one or more of the following positions on the basis of the domain 2 of the LAG-3 protein: 177, 183, 185, 186, 187, 190, 195, 197, 198, 199, 201, 207, 212, 214 and 218, or preferably, mutations at one or more of the following positions on the basis of the domain 2 of the LAG-3 protein: 183, 185, 186, 187, 190, 195, 197, 199, 201, 207 and 212, wherein the numbering of the amino acid positions corresponds to the numbering of the sequence set forth in SEQ ID NO: 63, and preferably, the sequence of the domain 2 of the LAG-3 protein is set forth in SEQ ID NO: 11; preferably, the LAG-3 protein comprises the domain 1 and the domain 2, and optionally the domain 3 and/or the domain 4;
preferably, the LAG-3 protein comprises the intact LAG-3 protein or an LAG-3 protein fragment, wherein the LAG-3 protein fragment is selected from the group consisting of:
(1) an LAG-3 protein fragment comprising or consisting of domain 1 and domain 2;
(2) an LAG-3 protein fragment comprising or consisting of domain 1, domain 2, and domain
3; or
(3) an LAG-3 protein fragment comprising or consisting of domain 1, domain 2, domain 3, and domain 4.

2. The LAG-3 protein mutant according to item 1, comprising one or more of the following mutations on the basis of the domain 2 of the LAG-3 protein: R188A, R192A, H196A, H197A, P172A, P175A, S177A, V178A, N183A, G185A, Q186A, G187A, V189A, P190A, P195A, L199A, F203A, Q208A, S210A, P211A, M212A, S214A, P216A, G218A, H198G, H198L, H198M, H198W, H198Y, H198V, E201R, E201N, E201D, E201Q, E201H, E201G, E201F, E201S, P207R, P207D, P207E, P207I, P207M, P207S, P207T, P207Y and V209T, preferably, P207E, P207I, P207R, P207D, M212A, P207M, S177A, P207T, Q186A, G187A, E201D, E201G, H197A, H198Y, G185A, L199A, N183A, P190A, P195A, S214A, P207Y, G218A, H198W and H198V, or preferably, N183A, G185A, Q186A, G187A, P190A, P195A, H197A, L199A, E201D, E201G, P207E, P207I, P207R, P207D, M212A and P207M.

3. The LAG-3 protein mutant according to item 1 or 2, wherein the mutation present in the domain 2 of the LAG-3 protein is selected from the group consisting of: R188A; R192A; H196A; H197A; P172A; P175A; S177A; V178A; N183A; G185A; Q186A; G187A; V189A; P190A; P195A; L199A; F203A; Q208A; S210A; P211A; M212A; S214A; P216A; G218A; H198G; H198L; H198M; H198W; H198Y; H198V; E201R; E201N; E201D; E201Q; E201H; E201G; E201F; E201S; P207R; P207D; P207E; P207I; P207M; P207S; P207T; P207Y; V209T; P207E and M212A; P207E and E201D; P207I and E201G; E201D and Q186A; H197A and E201G; P207I, E201D and Q186A; E201D, Q186A and P195A; P207E, Q186A and E201G; P207E, E201D, P195A and H197A; P207I, M212A, E201D and N183A; and P207E, M212A, E201G and N183A, N183A, G185A, Q186A, G187A, P190A, P195A, L199A and E201D; or preferably, the mutation is selected from the group consisting of: N183A; G185A; Q186A; G187A; P190A; P195A; L199A; E201D; E201G; P207E and M212A; P207E and E201D; P207I and E201G; E201D and Q186A; H197A and E201G; and P207I, E201D and Q186A;
preferably, the domain 2 of the LAG-3 protein mutant comprises a sequence set forth in any one of SEQ ID NOs: 14-60.

4. An LAG-3 fusion protein, comprising a structure as follows: a structural unit 1—a structural unit 2, wherein the structural unit 1 is selected from LAG-3 D1-D2, LAG-3 D1-D2-D3, or LAG-3 D1-D2-D3-D4,
wherein D1 denotes the domain 1 of the LAG-3, D2 denotes the domain 2 of the LAG-3 protein or a domain 2 mutant, D3 denotes the domain 3 of the LAG-3, and D4 denotes the domain 4 of the LAG-3;
preferably, D1 has a sequence set forth in SEQ ID NO: 10 or set forth in amino acids 37-167 of SEQ ID NO: 64;
D2 has a sequence set forth in SEQ ID NO: 11, set forth in the sequence of the domain 2 of the LAG-3 protein mutant according to any one of items 1-3, or set forth in amino acids 168-252 of SEQ ID NO: 64;
D3 has a sequence set forth in SEQ ID NO: 12 or set forth in amino acids 265-343 of SEQ ID NO: 64;
D4 has a sequence set forth in SEQ ID NO: 13 or set forth in amino acids 348-419 of SEQ ID NO: 64;
the structural unit 2 is a structural unit enabling the LAG-3 fusion protein to form a dimer or multimer, and is preferably selected from: an Fc fragment (preferably the Fc region is an Fc region from an IgG (e.g., IgG1, IgG2, IgG3 or IgG4) antibody, preferably having a sequence set forth in SEQ ID NO: 1); a VL-CL or VH-CH1 of an Fab fragment, wherein the VL-CL and VH-CH1 are paired to form an Fab fragment or Fab′ fragment specific for an antigen (preferably when the VL-CL of the Fab fragment has a sequence set forth in SEQ ID NO: 4, the VH-CH1 has a sequence set forth in SEQ ID NO: 5; or when the VL-CL has a sequence set forth in SEQ ID NO: 61, the VH-CH1 has a sequence set forth in SEQ ID NO: 62); or a c-JUN (preferably having a sequence set forth in positions 1-39 of SEQ ID NO: 2) or c-FOS (preferably having a sequence set forth in positions 1-39 of SEQ ID NO: 3), wherein the c-JUN and c-FOS are paired to form a leucine zipper; when D2 denotes the D2 domain of the LAG-3 protein, the structural unit 2 is either VL-CL or VH-CH1 of the Fab fragment.

5. An LAG-3 fusion protein dimer or multimer, comprising the LAG-3 fusion protein according to item 4, wherein the structural unit 1 in the LAG-3 fusion protein dimer or multimer is identical or different.

6. The LAG-3 fusion protein dimer or multimer according to item 5, wherein the LAG-3 fusion protein dimer or multimer is an LAG-3 fusion protein dimer, and the structural unit 1 is selected from LAG-3 D1-D2, LAG-3 D1-D2-D3, or LAG-3 D1-D2-D3-D4,
wherein D1 denotes the domain 1 of the LAG-3, D2 denotes the domain 2 of the LAG-3 protein or a domain 2 mutant, D3 denotes the domain 3 of the LAG-3, and D4 denotes the domain 4 of the LAG-3;
preferably, D1 has a sequence set forth in SEQ ID NO: 10 or set forth in amino acids 37-167 of SEQ ID NO: 64;
D2 has a sequence set forth in SEQ ID NO: 11, set forth in the sequence of the domain 2 of the LAG-3 protein mutant according to any one of items 1-3, or set forth in amino acids 168-252 of SEQ ID NO: 64;
D3 has a sequence set forth in SEQ ID NO: 12 or set forth in amino acids 265-343 of SEQ ID NO: 64;
D4 has a sequence set forth in SEQ ID NO: 13 or set forth in amino acids 348-419 of SEQ ID NO: 64;
wherein the structural unit 2 is selected from:
(1) an Fc fragment, wherein preferably, the Fc fragment has a sequence set forth in SEQ ID NO: 1; or
(2) a VL-CL or VH-CH1, wherein the VL-CL and VH-CH1, as two structural units 2 in the LAG-3 fusion protein dimer, are paired to form an Fab fragment specific for an antigen; preferably the antigen is selected from a tumor cell surface antigen, an immune cell surface antigen, a virus, a bacterium, an endotoxin, or a cytokine, e.g., CD3, SLAMF7, CD38, BCMA, CD16a, CEA, PD-L1, PD-1, CTLA-4, TIGIT, LAG-3, VEGF, B7-H3, TGF-β or IL-10; preferably, the VL-CL of the Fab fragment has a sequence set forth in SEQ ID NO: 4, and the VH-CH1 has a sequence set forth in SEQ ID NO: 5.

7. The LAG-3 fusion protein according to item 4, wherein the LAG-3 D1, D2, D3, D4, and the structural unit 2 are connected with or without a linker, and preferably the linker is selected from a sequence set forth in any one of SEQ ID NOs: 6-9.

In some embodiments, the linker is flexible. In some other embodiments, the linker is rigid. In some embodiments, the linker may be a linker derived from a native multidomain protein or a linker peptide conventionally used in the art. In some embodiments, the linker may be designed using a linker design database and a computer program.

8. A conjugate, comprising the LAG-3 protein mutant according to any one of items 1-3 and a conjugated moiety, or comprising the LAG-3 fusion protein according to item 4 and a conjugated moiety, or comprising the LAG-3 fusion protein dimer or multimer according to item 5 or 6 and a conjugated moiety, wherein the conjugated moiety is a purification tag (e.g., His-tag, Fc-tag), a detectable label, a drug, a prodrug, a toxin, a cytokine, a protein (e.g., an enzyme), a virus, a lipid, a biological response modulator (e.g., an immunomodulator), PEG, a hormone, a polypeptide, an oligonucleotide, a diagnostic agent, a cytotoxic agent, or a combination thereof, preferably, the conjugated moiety is a radioisotope, a fluorescent substance, a chemiluminescent substance, a colored substance, a chemotherapeutic agent, a biotoxin, polyethylene glycol, or an enzyme.

9. A pharmaceutical composition, comprising the LAG-3 protein mutant according to any one of items 1-3, or the fusion protein according to item 4, or the LAG-3 fusion protein dimer or multimer according to item 5 or 6, or the conjugate according to item 8, wherein preferably, the pharmaceutical composition further comprises at least one drug for treating a cancer or an infectious disease; preferably the drug is selected from a chemotherapeutic drug, an immunotherapeutic drug, or a combination thereof; preferably, the drug is selected from a radiotherapeutic agent, a chemotherapeutic agent (e.g., paclitaxels, anthracyclines, gemcitabine), a therapeutic antibody (e.g., rituximab, cetuximab, edrecolomab, trastuzumab, an anti-PD-1 antibody, an anti-PD-L1 antibody), a cytokine, a polypeptide, an antimetabolite, or a combination thereof;
preferably, the pharmaceutical composition further comprises at least one immune checkpoint regulator selected from: (a) an antagonist of an inhibitory immune checkpoint molecule; and (b) an agonist of a stimulatory immune checkpoint molecule.

10. Use of the LAG-3 protein mutant according to any one of items 1-3, or the fusion protein according to item 4, or the LAG-3 fusion protein dimer or multimer according to item 5 or 6, or the conjugate according to item 8 in modulating an immune response or immunostimulation or treating or diagnosing a cancer or Parkinson's disease, or in preparing a medicament, an immunostimulant or an adjuvant for modulating an immune response or treating or diagnosing a cancer or Parkinson's disease.

In some embodiments, the pharmaceutical composition is administered in combination, preferably simultaneously or sequentially, with additional therapeutic or prophylactic regimens, e.g., radiation therapy, chemotherapy, or immunotherapy.

In some embodiments, preferably, the chemotherapeutic agent is an alkylating agent, an antimetabolite, an antibiotic, a botanical agent, and/or a hormonal agent, preferably cyclophosphamide, pemetrexed, a platinum-based drug such as cisplatin, carboplatin or oxaliplatin, doxorubicin, paclitaxel, a vinca alkaloid, an anthracycline, gemcitabine, tamoxifen, megestrol, goserelin, asparaginase, and/or a fluorouracil antineoplastic agent.

11. A nucleic acid molecule, comprising a nucleic acid sequence encoding the LAG-3 protein mutant according to any one of items 1-3, or the fusion protein according to item 4, or the LAG-3 fusion protein dimer or multimer according to item 5 or 6, or the conjugate according to item 8, or a complement thereof.

12. A vector, comprising the nucleic acid molecule according to item 11.

13. A host cell, comprising the nucleic acid molecule according to item 11 or the vector according to item 12.

14. A method of treating a disease, comprising administering to a subject in need a therapeutically effective amount of the LAG-3 protein mutant according to any one of items 1-3, or the fusion protein according to item 4, or the LAG-3 fusion protein dimer or multimer according to item 5 or 6, or the conjugate according to item 8, or the pharmaceutical composition according to item 9.

15. A kit, comprising the LAG-3 protein mutant according to any one of items 1-3, or the fusion protein according to item 4, or the LAG-3 fusion protein dimer or multimer according to item 5 or 6, or the conjugate according to item 8, or the pharmaceutical composition according to item 9, wherein preferably, the kit further comprises an antibody that specifically recognizes the LAG-3 protein; optionally, the antibody further comprises a detectable label, such as a radioisotope, a fluorescent substance, a chemiluminescent substance, a colored substance, or an enzyme.

It will be appreciated that within the scope of the present invention, the above technical features of the present invention and the technical features specifically described hereinafter (as in the examples) may be combined with each other to constitute a new or preferred embodiment. Due to the limited space, such embodiments are not described herein. The terms referred to in the present invention have the conventional meanings understood by those skilled in the art. Where a term has two or more definitions as used and/or acceptable in the art, the definitions of the terms used herein are intended to include all meanings.

The LAG-3 molecule consists of 3 moieties: an extracellular region, a transmembrane region, and an intracellular region. The extracellular region consists of 4 immunoglobulin domains: D1 (the domain 1 of the LAG-3 protein), D2 (the domain 2 of the LAG-3 protein), D3 (the domain 3 of the LAG-3 protein), and D4 (the domain 4 of the LAG-3 protein). The D1 region is a member of the V immunoglobulin superfamily (IgSF), and the D2, D3 and D4 regions are members of the C2 IgSF. The D1 domain comprises an extra loop consisting of 30 amino acids rich in proline, which is reported to be involved in the interaction between LAG-3 and major histocompatibility complex class II (MHCII). In 2019, Chen Lieping et al. found that FGL1 is a ligand of LAG-3 for the T cell inhibitory function, and through deletion of specific domains, it was demonstrated that the D1 and D2 of the LAG-3 are the major domains interacting with FGL1 (Wang et al., Fibrinogen-like Protein 1 Is a Major Immune Inhibitory Ligand of LAG-3, Cell (2019)). The LAG-3 binds to MHCII and FGL1 dependent on the D1 and D2. It will be appreciated by those of ordinary skills in the art that a partial or intact LAG-3 protein comprising the D1 and D2 domains of the LAG-3 may achieve the interaction with FGL1 or MHCII, e.g., a partial LAG-3 protein comprising the D1, D2 and D3 domains, or an intact LAG-3 protein comprising the D1, D2, D3 and D4 domains.

In one embodiment of the present invention, the D1 has a sequence set forth in SEQ ID NO: 10; the D2 has a sequence set forth in SEQ ID NO: 11 or set forth in the domain 2 mutant of the present invention, the domain 2 mutant being defined as the domain 2 of the LAG-3 protein mutant according to any one of items 1-3; the D3 has a sequence set forth in SEQ ID NO: 12; and the D4 has a sequence set forth in SEQ ID NO: 13.

In one embodiment of the present invention, the D1-D4 domains of the LAG-3 are defined according to the Uniprot database, the sequences of the D1, D2, D3 and D4 of the LAG-3 differ from the D1, D2, D3 and D4 sequences of the present invention (SEQ ID NOs: 10, 11, 12 and 13) in the N and C termini, as seen in Uniprot P18627 (SEQ ID NO: 64), wherein the sequence definitions of D1-D4 are as follows: D1, amino acids 37-167; D2, amino acids 168-252; D3, amino acids 265-343; D4, amino acids 348-419. It will be appreciated by those of ordinary skills in the art that the D1-D4 domains of the LAG-3 protein defined according to the Uniprot database, exert the same function as the corresponding D1-D4 (SEQ ID NOs: 10-13) of the present invention.

LAG-3 (CD223) is known to induce the maturation of monocyte-derived dendritic cells in vitro and to induce CD41 helper T cell responses and CD8 T cell responses in vivo as an immunotherapeutic adjuvant. Further information on LAG-3 and use thereof as an immunostimulant can be found in the works of TRIEBELE et al., TRIEBEL et al., and HUARD et al. Some soluble forms of LAG-3 are capable of binding to MHCII molecules and inducing dendritic cell maturation and migration to secondary lymphoid organs where they are capable of initiating naive CD4⁻ helper cells and CD8 cytotoxic T cells leading to tumor rejection. Recently, a recombinant soluble human LAG-3-Ig fusion protein has been demonstrated to activate a wide range of effector cells, e.g., inducing monocyte-macrophages to secrete cytokines/chemokines, in both innate and acquired immune responses.

In one preferred embodiment of the present invention, the LAG-3 protein fragment is selected from any one of the following:
A) a full-length soluble fragment of the native LAG-3 protein, wherein the soluble fragment retains a domain of the LAG-3 protein having the capacity of binding to a natural ligand of the LAG-3 protein or part of the extracellular segment of the LAG-3 protein, but lacks part or all of the transmembrane segment and the intracellular segment of the LAG-3 protein;
B) a fragment comprising the full length of the extracellular segment of the native LAG-3 protein;
C) a fragment comprising the extracellular segment of the native LAG-3 protein and retains the biological activity of the extracellular segment;
D) an extracellular segment of the native LAG-3 protein with the removal of one or more (for example, 5-10) continuous amino acid residues at the N terminus, the C terminus, or both;
E) a mutant of the LAG-3 protein.

In the present invention, the structural units for forming the dimer may be selected from, for example, an Fc fragment, c-JUN, c-FOS, VL-CL, and VH-CH1, wherein the VL-CL and VH-CH1 are paired to form an Fab fragment specific for an antigen, and the c-JUN and c-FOS are paired to form a leucine zipper.

In a specific embodiment, the Fc fragment has a sequence set forth in SEQ ID NO: 1; when the VL-CL has a sequence set forth in SEQ ID NO: 4, the VH-CH1 has a sequence set forth in SEQ ID NO: 5, or when the VL-CL has a sequence set forth in SEQ ID NO: 61, the VH-CH1 has a sequence set forth in SEQ ID NO: 62; the c-JUN-His has a sequence set forth in SEQ ID NO: 2, and the c-FOS-His has a sequence set forth in SEQ ID NO: 3.

In the present invention, the structural unit for forming a trimer is the T4 fibritin foldon domain.

It will be appreciated by those of ordinary skills in the art that structural units capable of being used to form a dimer or multimer known in the art can all be used to form the dimer or multimer of the present invention.

In some embodiments, the LAG-3 protein mutant, LAG-3 fusion protein, or LAG-3 fusion protein dimer or multimer of the present invention may be conjugated with a therapeutic agent, a prodrug, a peptide, a protein, an enzyme, a virus, a lipid, a biological response modifier, a pharmaceutical agent or PEG. The LAG-3 protein mutant, LAG-3 fusion protein, or LAG-3 fusion protein dimer or multimer of the present invention can be linked or fused to a therapeutic agent, and the therapeutic agent may comprise a detectable label such as a radioactive label, an immunomodulator, a hormone, an enzyme, a polypeptide, an oligonucleotide, a photoactive therapeutic or diagnostic agent, a cytotoxic agent that may be a drug or a toxin, an ultrasound enhancing agent, a nonradioactive label, a combination thereof and other such ingredients known in the art.

In the present invention, linker denotes linker, linker1 denotes linker1, linker2 denotes linker2, and linker3 denotes linker 3.

Compared with the prior art, the present invention has one or more of the following beneficial effects:

The LAG-3 protein mutant features a high expression level, improved purity, excellent biological activity and specificity, significant in-vitro and in-vivo anti-tumor bioactivity, and good stability. The dimer with the LAG-3-Fab structure (such as LAG3 D1-D2-D3-D4-Fab and LAG3 D1-D2-Fab) of the present invention retains the activity of the LAG-3 moiety and the Fab moiety, and has good stability.

### BRIEF DESCRIPTION OF THE DRAWINGS

FIG. 1 is an illustrative schematic of LAG-3-Fc. A, LAG-3 D1-D2-Fc; B, LAG-3 D1-D2-D3-D4-Fc.

FIG. 2 illustrates the amino acid sequence of LAG-3 D1-D2 (wild type), wherein amino acids 1-149 are the D1 domain and 150-239 are the D2 domain (in italic and bold font).

FIG. 3 illustrates the ELISA result of LAG-3 D1-D2-Fc wild type and mutants thereof binding to human FGL1 (hFGL1).

FIG. 4 illustrates the FACS result of LAG-3 D1-D2-Fc wild type and mutants thereof binding to human MHCII Daudi cells, wherein A, direct binding; B, competitive binding with IMP321.

FIG. 5 illustrates the non-reduced SDS-PAGE result of thermally accelerated samples of LAG-3 D1-D2-Fc wild type and mutant thereof, wherein lane 1, W1161-WT D0; lane 2, W1161-WT D7; lane 3, W1161-WT D14; lane 4, WS447 D0; lane 5, WS447 D7; lane 6, WS447 D14; MK, protein marker.

FIG. 6 illustrates the ELISA result of LAG-3 D1-D2-D3-D4-Fc wild type and mutant thereof binding to human FGL1 (hFGL1).

FIG. 7 illustrates the FACS result of LAG-3 D1-D2-D3-D4-Fc wild type and mutants thereof direct binding to human MHCII⁺ Daudi cells.

FIG. 8 illustrates the non-reduced SDS-PAGE result of thermally accelerated samples of LAG-3 D1-D2-D3-D4-Fc wild type and mutant thereof, wherein lane 1, IMP321 D0; lane 2, IP321 D14; lane 3, A7817 D0; lane 4, A7817 D14; MK, protein marker.

FIG. 9 is an illustrative schematic of LAG-3 D1-D2-LZ, wherein LZ denotes a leucine zipper.

FIG. 10 illustrates the ELISA result of LAG-3 D1-D2-LZ wild type and mutants thereof binding to human FGL1 (hFGL1).

FIG. 11 illustrates the FACS result of LAG-3 D1-D2-LZ wild type and mutants thereof direct binding to human MHCII Daudi cells.

FIG. 12 is an illustrative schematic of LAG-3-Fab. The LAG-3 includes two forms: D1-D2 and D1-D2-D3-D4.

FIG. 13 illustrates the non-reduced SDS-PAGE result of thermally accelerated samples of LAG-3-Fab. Lane 1, Y103-7A D0; lane 2, Y103-4A D0; lane 3, Y103-7A D14; lane 4, Y103-4A D14.

FIG. 14 illustrates the bioactivity result of LAG-3 D1-D2-Fc and LAG-3 D1-D2-D3-D4-Fc mutants in inducing JAWSII cells to release mTNF-α.

### DETAILED DESCRIPTION

The embodiments of the present invention will be described in detail below with reference to the examples. Those skilled in the art will appreciate that the following examples are only for illustrating the present invention, and should not be construed as limitations to the scope of the present invention. This present invention may be implemented in many other ways than those described herein, and it will be apparent to those skilled in the art that similar modifications can be made without departing from the spirit of the present invention. Therefore, the protection scope of the present invention is defined by the claims, rather than limited by the specific examples disclosed below.

### Example 1: LAG-3 D1-D2-Fc Wild Type and Mutants Thereof

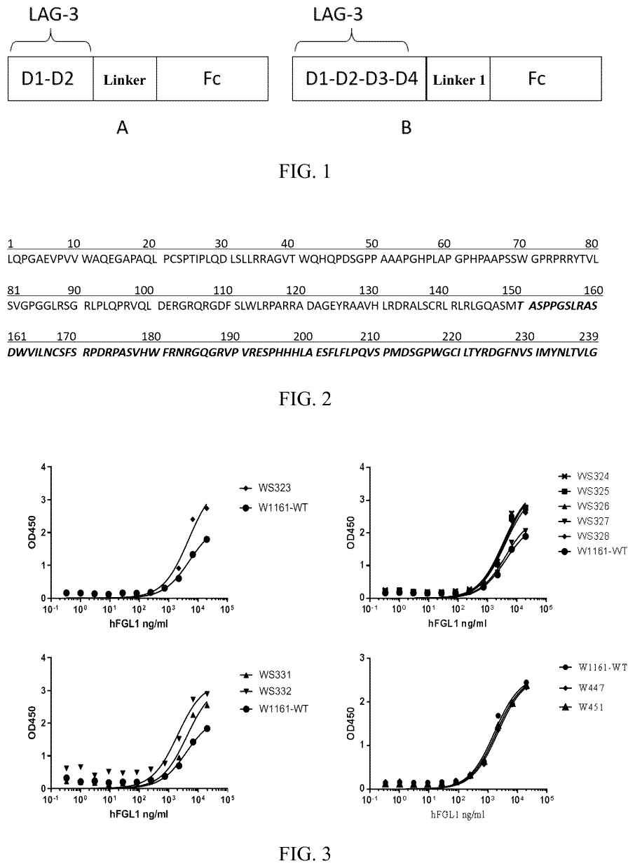

Gene fragments of the D1 and D2 domains of the LAG-3 were acquired by genetic synthesis. The Fc gene was added to the 3′ moiety of the D2 domain by a linker and the gene of LAG-3 D1-D2-Fc was inserted between polyclonal enzyme cleavage sites of eukaryotic expression vector pCDNA3.1 to give the corresponding eukaryotic expression vector (as shown in FIG. 1A). The corresponding amino acid sequence (SEQ ID NO: 63) and position of the LAG-3 D1-D2 domains are shown in FIG. 2. The D2 domain was subjected to amino acid point mutation to give different mutants. The specific structure of LAG-3 D1-D2-Fc and mutant constructs thereof was D1-D2-Linker-Fc. The sequence information is shown in Table 1.

TABLE 1
Amino acid sequence information of
LAG-3 D1-D2-Fc and mutants thereof

| Construct | Mutation | Sequence | | | |
|---|---|---|---|---|---|
| number | site | D1 | D2 | Linker | Fc |
| W1161-WT | Wild type | SEQ ID | SEQ ID NO: 11 | SEQ ID | SEQ ID |
| WS122 | R188A | NO: 10 | SEQ ID NO: 14 | NO: 6 | NO: 1 |
| WS123 | R192A | | SEQ ID NO: 15 | | |
| WS125 | H196A | | SEQ ID NO: 16 | | |
| WS126 | H197A | | SEQ ID NO: 17 | | |
| WS317 | P172A | | SEQ ID NO: 18 | | |
| WS318 | P175A | | SEQ ID NO: 19 | | |
| WS319 | S177A | | SEQ ID NO: 20 | | |
| WS320 | V178A | | SEQ ID NO: 21 | | |
| WS323 | N183A | | SEQ ID NO: 22 | | |
| WS324 | G185A | | SEQ ID NO: 23 | | |
| WS325 | Q186A | | SEQ ID NO: 24 | | |
| WS326 | G187A | | SEQ ID NO: 25 | | |
| WS327 | V189A | | SEQ ID NO: 26 | | |
| WS328 | P190A | | SEQ ID NO: 27 | | |
| WS331 | P195A | | SEQ ID NO: 28 | | |
| WS332 | L199A | | SEQ ID NO: 29 | | |
| WS334 | F203A | | SEQ ID NO: 30 | | |
| WS339 | Q208A | | SEQ ID NO: 31 | | |
| WS341 | S210A | | SEQ ID NO: 32 | | |
| WS342 | P211A | | SEQ ID NO: 33 | | |
| WS343 | M212A | | SEQ ID NO: 34 | | |
| WS344 | S214A | | SEQ ID NO: 35 | | |
| WS346 | P216A | | SEQ ID NO: 36 | | |
| WS348 | G218A | | SEQ ID NO: 37 | | |
| WS433 | H198G | | SEQ ID NO: 38 | | |
| WS435 | H198L | | SEQ ID NO: 39 | | |
| WS437 | H198M | | SEQ ID NO: 40 | | |
| WS442 | H198W | | SEQ ID NO: 41 | | |
| WS443 | H198Y | | SEQ ID NO: 42 | | |
| WS444 | H198V | | SEQ ID NO: 43 | | |
| WS445 | E201R | | SEQ ID NO: 44 | | |
| WS446 | E201N | | SEQ ID NO: 45 | | |
| WS447 | E201D | | SEQ ID NO: 46 | | |
| WS449 | E201Q | | SEQ ID NO: 47 | | |
| WS450 | E201H | | SEQ ID NO: 48 | | |
| WS451 | E201G | | SEQ ID NO: 49 | | |
| WS456 | E201F | | SEQ ID NO: 50 | | |
| WS458 | E201S | | SEQ ID NO: 51 | | |
| WS482 | P207R | | SEQ ID NO: 52 | | |
| WS484 | P207D | | SEQ ID NO: 53 | | |
| WS486 | P207E | | SEQ ID NO: 54 | | |
| WS488 | P207I | | SEQ ID NO: 55 | | |
| WS491 | P207M | | SEQ ID NO: 56 | | |
| WS494 | P207S | | SEQ ID NO: 57 | | |
| WS495 | P207T | | SEQ ID NO: 58 | | |
| WS497 | P207Y | | SEQ ID NO: 59 | | |
| WS512 | V209T | | SEQ ID NO: 60 | | |

### Example 2: Expression and Purification of LAG-3 D1-D2-Fc Wild Type and Mutants Thereof

Plasmids were extracted according to a conventional plasmid extraction method and used for chemical transfection of CHO-S cells (gibco). The transfected cells were cultured on a shaker at 5% s CO₂/37° C. for 7-10 days. The supernatant was harvested by 3000×g centrifugation and filtered through a 0.22 m filter. The LAG-3 D1-D2-Fc wild type fusion protein and mutants thereof were obtained by purification through protein A affinity chromatography. The concentration of the purified protein was determined by UV absorbance at 280 nm and the corresponding extinction coefficient, and the expression level of the proteins was calculated. The expression level of some mutants was significantly higher than that of the wild type, as shown in Table 2.

TABLE 2
Expression of LAG-3 D1-D2-Fc wild type and mutants thereof

| Construct | Mutation | Expression |
|---|---|---|
| number | site | (mg/L) |
| W1161-WT | Wild type | 15 |
| WS122 | R188A | 27 |
| WS123 | R192A | 32 |
| WS125 | H196A | 40 |
| WS126 | H197A | 45 |
| WS317 | P172A | 30 |
| WS318 | P175A | 30 |
| WS319 | S177A | 57 |
| WS320 | V178A | 40 |
| WS323 | N183A | 30 |
| WS324 | G185A | 43 |
| WS325 | Q186A | 50 |
| WS326 | G187A | 47 |
| WS327 | V189A | 34 |
| WS328 | P190A | 38 |
| WS332 | L199A | 30 |
| WS334 | F203A | 30 |
| WS339 | Q208A | 35 |
| WS341 | S210A | 35 |
| WS342 | P211A | 30 |
| WS343 | M212A | 60 |
| WS344 | S214A | 47 |
| WS346 | P216A | 30 |
| WS348 | G218A | 45 |
| WS433 | H198G | 30 |
| WS435 | H198L | 35 |
| WS437 | H198M | 28 |
| WS442 | H198W | 45 |
| WS443 | H198Y | 45 |
| WS444 | H198V | 43 |
| WS445 | E201R | 32 |
| WS446 | E201N | 28 |
| WS447 | E201D | 37 |
| WS449 | E201Q | 30 |
| WS450 | E201H | 34 |
| WS451 | E201G | 32 |
| WS456 | E201F | 27 |
| WS458 | E201S | 27 |
| WS482 | P207R | 65 |
| WS484 | P207D | 64 |
| WS486 | P207E | 82 |
| WS488 | P207I | 69 |
| WS491 | P207M | 60 |
| WS495 | P207T | 54 |
| WS497 | P207Y | 47 |

The multimer content of the LAG-3 D1-D2-Fc wild type and the mutants thereof was determined by high performance size exclusion chromatography (HPLC-SEC). Compared with those of the wild type, some mutants demonstrated significantly reduced multimer content and improved purity, as shown in Table 3. SDS-PAGE showed that the molecular weight was consistent with the theoretical value 110 WD.

TABLE 3
HPLC-SEC purity of LAG-3 D1-D2-Fc
wild type and mutants thereof

| Construct | Mutation | |
|---|---|---|
| number | site | HPLC-SEC |
| W1161-WT | Wild type | 65.65% |
| WS126 | H197A | 87.40% |
| WS319 | S177A | 79.34% |
| WS323 | N183A | 84.27% |
| WS324 | G185A | 85.12% |
| WS325 | Q186A | 86.84% |
| WS326 | G187A | 86.21% |
| WS328 | P190A | 83.00% |
| WS331 | P195A | 81.07% |
| WS332 | L199A | 84.52% |
| WS339 | Q208A | 79.63% |
| WS341 | S210A | 82.49% |
| WS342 | P211A | 84.00% |
| WS343 | M212A | 84.79% |
| WS344 | S214A | 80.43% |
| WS346 | P216A | 80.65% |
| WS443 | H198Y | 86.37% |
| WS447 | E201D | 89.03% |
| WS451 | E201G | 87.73% |
| WS482 | P207R | 81.77% |
| WS488 | P207I | 78.50% |
| WS494 | P207S | 78.09% |
| WS495 | P207T | 85.02% |
| WS512 | V209T | 79.22% |

### Example 3: Activity Assay of LAG-3 D1-D2-Fc Wild Type and Mutants Thereof

1. Binding capacity of LAG-3 D1-D2-Fc wild type and mutant thereof to human FGL1 (hFGL1)

The binding capacity of the LAG-3 D1-D2-Fc wild type and the mutants thereof to human FGL1 (hFGL1) was determined by ELISA. The samples were prepared into 10 μg/mL coating solutions by using PBS buffer, added to a plate (100 μg/well), and incubated overnight at 4° C. The next day, the remaining coating solution was discarded. PBST (PBS containing 0.1% Tween 20) was added at 300 μL/well for washing once, and 3% BSA was added at 300 μL/well for blocking the wells at 37° C. for 1 h. After 1 wash with 300 μL/well of PBST, serially diluted human FGL1-His antigen (ACRO, FG1-H52Hy) solutions were added at 100 μL/well and the plate was incubated at 37° C. for 1 h. After 3 washes with 300 L/well of PBST, diluted 6×His tag antibody [GT359] (HRP) (GeneTex, GTX628914-01) was added at 100 μL/well and the plate was incubated at 37° C. for 1 h. After 5 washes with 300 μL/well of PBST, a TMB chromogenic solution was added (100 μL/well). Finally, the reaction was stopped by adding 2 M HCl and the samples were detected on a microplate reader (Molecular Devices, SPECTRA Max plus 384) for OD450.

The results are shown in FIG. 3. Compared with the wild type W1161-WT, the mutants WS323, WS324, WS325, WS326, WS328, WS331, and WS332 demonstrated improved binding capacity to human FGL1, while WS447 and WS451 remained unchanged.

2. Binding capacity of LAG-3 D1-D2-Fc wild type and mutant thereof to human MHCII The direct and competitive binding capacities of LAG-3 D1-D2-Fc wild type and mutants thereof to human MHCII were determined by FACS using Daudi cells (from CCTCC-GDC097) as positive cells of human MHCII.

Direct binding: Daudi cells were collected by centrifugation, resuspended in a buffer (PBS+1% FBS), and added at 1×10⁵ cells/100 μL/well in a 96-well plate. Then the mixture was centrifuged at 350×g for 5 min, and the supernatant was discarded. The samples were diluted to 2000 nM with a buffer and serially 3- or 4-fold diluted to the 11^{th} concentration. The samples were added to a 96-well plate at 100 μL/well, resuspended, incubated at 4° C. in the dark for 1 h, and centrifuged. The supernatant was discarded. The samples were washed twice with the buffer, resuspended in diluted PE-labeled anti-human IgG Fc antibody (Biolegend, 409304), incubated at 4° C. in the dark for 30 min, then washed twice with the buffer, resuspended in 100 μL of buffer, and loaded on a flow cytometer (BD Accuri™ C6) for detection.

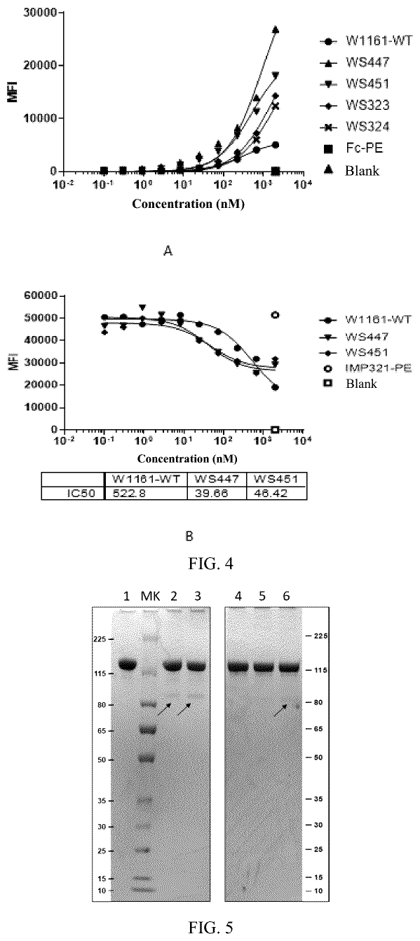

The results are shown in FIG. 4A. Compared with the wild type W1161-WT, the mutants WS323, WS324, WS447, and WS451 demonstrated improved direct binding capacity to human MHCII Daudi cells.

Competitive binding: Daudi cells were collected by centrifugation, resuspended in a buffer (PBS+1% FBS), and added at 1×10⁵ cells/100 μL/well in a 96-well plate. Then the mixture was centrifuged at 350×g for 5 min, and the supernatant was discarded. The samples were diluted to 4000 nM with a buffer and serially 3- or 4-fold diluted to the 11^{th} concentration. 30 μL of serially diluted sample was taken and mixed with an equal volume of 4000 nM IP321-PE (the IMP321 sequence is derived from SEQ ID NO: 17 in Patent No. US20110008331A1). The mixture was then added into a 96-well plate at 50 μL/well, resuspended, incubated in the dark for 30 min at 4° C., washed twice with a buffer, resuspended in 50 μL of buffer, and loaded on a flow cytometer (BD Accuri™ C6) for detection.

The results are shown in FIG. 4B. The mutant WS447 and WS451 were capable of competing with IMP321 for binding to MHCII Daudi cells with superior competitive binding capacity to that of the wild type W1161-WT.

Through the binding capacity to MHCII, FGL1 and the like, the mutants demonstrated significant in vitro and in vivo anti-tumor bioactivity.

### Example 4: Stability Test of LAG-3 D1-D2-Fc Wild Type and Mutants Thereof

The stability of the samples was evaluated using differential scanning calorimetry (DSC). Compared with the wild type W1161-WT, the mutants WS447 and WS451 demonstrated improved Tm1 values, as shown in Table 4.

TABLE 4
DSC results of LAG-3 D1-D2-Fc wild type and mutants thereof

| Construct number | Mutation site | Tm1 | |
|---|---|---|---|
| W1161-WT | Wild type | 56.4° | C. |
| WS447 | E201D | 58.59° | C. |
| WS451 | E201G | 57.89° | C. |

The samples were diluted to 0.5 mg/mL, added into 1.5-mL EP tubes at 100 μL/tube, and incubated in a water bath at 40° C. for thermal acceleration for 14 days. The start of the test was designated as D0, the 7^{th} day was designated as D7, and the 14^{th} day was designated as D14. After two weeks, the D0, D7, and D14 samples were subjected to SDS-PAGE.

As shown in FIG. 5, on D7 of thermal acceleration, a remarkable impurity band of about 80 kD was found in the wild type W1161-WT, while no impurity band was seen in the mutant WS447 on D7; on D14 of thermal acceleration, the impurity band signals of the mutant WS447 were significantly weaker than the wild type, indicating that the mutant WS447 has better stability than the wild type.

### Example 5: Preparation of LAG-3 D1-D2-D3-D4-Fc Wild Type and Mutants Thereof

The specific structure of LAG-3 D1-D2-D3-D4-Fc (FIG. 1B) and mutant constructs thereof was D1-D2-D3-D4-Linker1-Fc. The sequence information is shown in Table 5-1. The expression and purification method were the same as in Example 2. The multimer content was determined by high performance size exclusion chromatography (HPLC-SEC).

The results are shown in Table 5-2. The mutants A7817, A7820, A7836, and A7842 demonstrated improved expression and purity as compared with the wild type. The mutations E201D, E201G, P207I, and M212A produced similar effect on the domain D2 in the LAG-3 D1-D2-D3-D4-Fc fusion protein (A7817, A7820, A7836, and A7842) as the effect in the LAG-3 D1-D2-Fc fusion protein (WS447, WS451, WS488, and WS343). Compared with the wild type, the expression and purity were significantly improved. Similarly, for the LAG-3 D1-D2-D3-D4-Fc fusion protein, other mutations in the D2 domain (such as N183A, G185A, Q186A, G187A, H197A, H198Y, L199A, P207R, P207T, M212A, P211A, etc.) also increased the expression or purity compared with the wild type, and the effect (improvement in expression or purity) was similar to that produced in the LAG-3 D1-D2-Fc fusion protein.

TABLE 5-1
Amino acid sequence information of LAG-3
D1-D2-D3-D4-Fc and mutants thereof

| Construct | Mutation | Sequence | | | | | |
|---|---|---|---|---|---|---|---|
| number | site | D1 | D2 | D3 | D4 | Linker1 | Fc |
| IMP321 | Wild type | SEQ ID | SEQ ID | SEQ ID | SEQ ID | SEQ ID | SEQ ID |
| | | NO: 10 | NO: 11 | NO: 12 | NO: 13 | NO: 7 | NO: 1 |
| A7817 | E201D | | SEQ ID | | | | |
| | | | NO: 46 | | | | |
| A7820 | E201G | | SEQ ID | | | | |
| | | | NO: 49 | | | | |
| A7836 | P207I | | SEQ ID | | | | |
| | | | NO: 55 | | | | |
| A7842 | M212A | | SEQ ID | | | | |
| | | | NO: 34 | | | | |
| A7848 | N183A | | SEQ ID | | | | |
| | | | NO: 22 | | | | |
| A7850 | P195A | | SEQ ID | | | | |
| | | | NO: 28 | | | | |

TABLE 5-2
Expression and purity information of LAG-3
D1-D2-D3-D4-Fc and mutants thereof

| Construct number | Mutation site | Expression (mg/L) | HPLC-SEC |
|---|---|---|---|
| IMP321 | Wild type | 20 | 76.21% |
| A7817 | E201D | 47 | 85.72% |
| A7820 | E201G | 42 | 88.89% |
| A7836 | P207I | 76 | 82.33% |
| A7842 | M212A | 82 | 85.47% |

### Example 6: Activity Assay of LAG-3 D1-D2-D3-D4-Fc Wild Type and Mutants Thereof

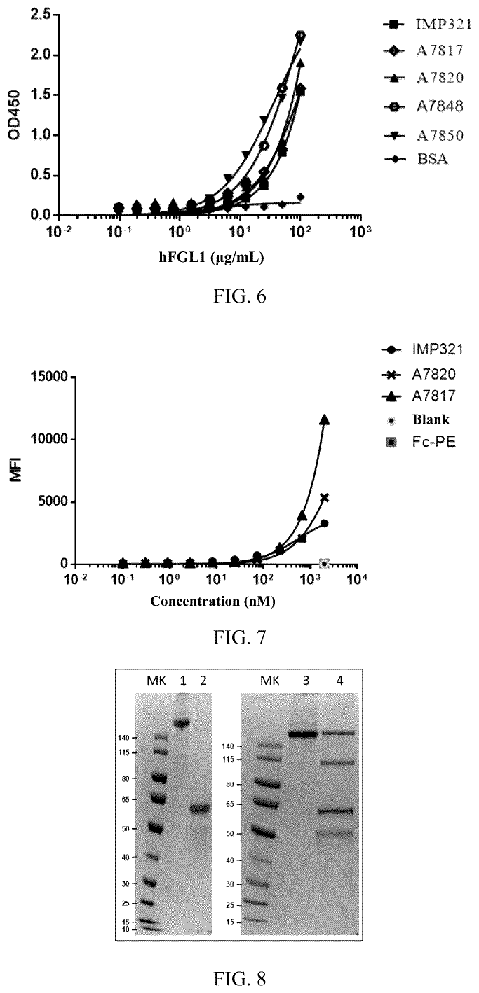

The binding capacity of the LAG-3 D1-D2-D3-D4-Fc wild type and the mutants thereof to human FGL1 was determined by ELISA using the same method as in Example 3. As shown in FIG. 6, compared with the IMP321 wild type, the binding capacity of mutants A7817 and A7820 to human FGL1 remained unchanged, and the binding capacity of mutants A7848 and A7850 to human FGL1 was improved. Similarly, for the LAG-3 D1-D2-D3-D4-Fc fusion protein, other mutations in the D2 domain (such as N183A, G185A, Q186A, G187A, P190A, P195A, L199A, etc.) also increased the binding capacity to human FGL1 compared with the wild type, and the same mutation site basically produced a similar change trend (change in binding capacity to human FGL1) in the LAG-3 D1-D2-D3-D4-Fc as that in the LAG-3 D1-D2-Fc.

The direct binding capacity of LAG-3 D1-D2-D3-D4-Fc wild type and mutants thereof to human MHCII was determined by FACS using Daudi cells as positive cells of human MHCII using the same method as in Example 3. As shown in FIG. 7, compared with the IMP321 wild type, the mutants A7817 and A7820 demonstrated improved direct binding capacity to human MHCII Daudi cells. Similarly, for the LAG-3 D1-D2-D3-D4-Fc fusion protein, other mutations in the D2 domain (such as N183A and G185A) also increased the binding capacity to human MHCII compared with the wild type, and the same mutation site basically produced a similar change trend (change in binding capacity to human MHCII) in the LAG-3 D1-D2-D3-D4-Fc as that in the LAG-3 D1-D2-Fc.

Through the binding capacity to MHCII, FGL1 and the like, the mutants demonstrated significant in vitro and in vivo anti-tumor bioactivity.

### Example 7: Stability Test of LAG-3 D1-D2-D3-D4-Fc Wild Type and Mutants Thereof

The stability of the samples was evaluated using differential scanning calorimetry (DSC). Compared with the wild type IMP321, the mutants A7817 and A7820 demonstrated improved Tm1 values, as shown in Table 6.

TABLE 6
DSC results of LAG-3 D1-D2-D3-D4-Fc
wild type and mutants thereof

| Construct number | Mutation site | Tm1 |
|---|---|---|
| IMP321 | Wild type | 52.09° C. |
| A7817 | E201D | 55.67° C. |
| A7820 | E201G | 54.79° C. |

The samples were diluted to 0.5 mg/mL, added into 1.5-mL EP tubes at 100 μL/tube, and incubated in a water bath at 40° C. for thermal acceleration for 14 days. The start of the test was designated as D0, and the 14^{th} day was designated as D14. After two weeks, the D0 and D14 samples were subjected to SDS-PAGE. As shown in FIG. 8, on D14 of thermal acceleration, the IMP321 wild type was completely degraded, while the mutant A7817 was only partially degraded with clear target bands, demonstrating that the stability of mutant A7817 was better than that of the wild type. The same mutation site basically produced a similar change trend (change in thermal stability) in the LAG-3 D1-D2-D3-D4-Fc as that in the LAG-3 D1-D2-Fc.

### Example 8: Preparation of LAG-3 D1-D2-LZ Wild Type and Mutants Thereof

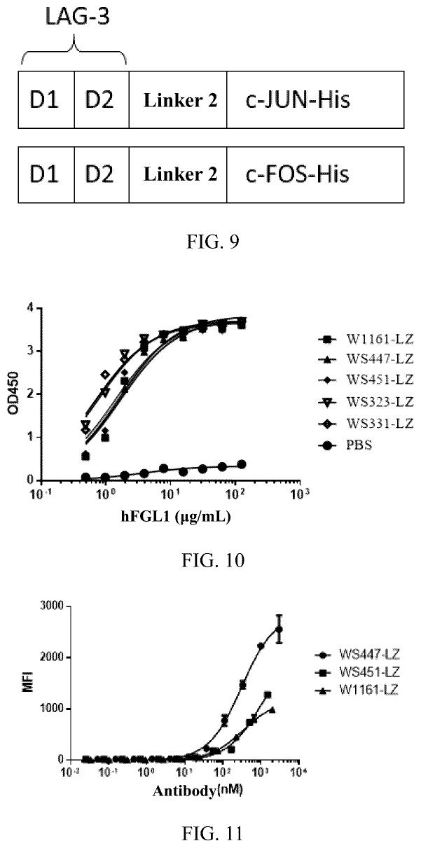

A schematic of the LAG-3 D1-D2-LZ is shown in FIG. 9. The specific structures of the wild type and the mutant constructs were D1-D2-Linker2-c-JUN-His and D1-D2-Linker2-c-FOS-His, wherein the c-JUN-His and the c-FOS-His can be paired with each other to enable the LAG-3 D1-D2-LZ to form a dimeric structure. The sequence information is shown in Table 7-1. The expression method is the same as in Example 2. The proteins were purified by affinity chromatography on a Ni column and the multimer content was determined by high performance size exclusion chromatography (HPLC-SEC).

The results are shown in Table 7-2. The expression and purity of the mutants WS447-LZ, WS451-LZ, WS488-LZ, and WS343-LZ were improved as compared with the wild type. Furthermore, the mutations E201D, E201G, P207I, and M212A produced similar effect on the domain D2 in the LAG-3 D1-D2-LZ fusion protein (WS447-LZ, WS451-LZ, WS488-LZ, and WS343-LZ) as the effect in the LAG-3 D1-D2-Fc fusion protein (WS447, WS451, WS488, and WS343). Compared with the wild type, the expression and purity were significantly improved.

Similarly, for the LAG-3 D1-D2-LZ fusion protein, other mutations in the D2 domain (such as N183A, G185A, Q186A, G187A, H197A, H198Y, L199A, P207R, P207T, M212A, P211A, etc.) also increased the expression or purity compared with the wild type, and the effect (improvement in expression or purity) was similar to that produced in the LAG-3 D1-D2-Fc fusion protein.

TABLE 7-1
Amino acid sequence information of LAG-3 D1-D2-LZ and mutants thereof

| Construct | Mutation | Sequence | | | | |
|---|---|---|---|---|---|---|
| number | site | D1 | D2 | Linker2 | c-JUN-His | c-FOS-His |
| W1161-LZ | Wild type | SEQ ID | SEQ ID NO: 11 | SEQ ID | SEQ ID | SEQ ID |
| WS447-LZ | E201D | NO: 10 | SEQ ID NO: 46 | NO: 8 | NO: 2 | NO: 3 |
| WS451-LZ | E201G | | SEQ ID NO: 49 | | | |
| WS488-LZ | P207I | | SEQ ID NO: 55 | | | |
| WS343-LZ | M212A | | SEQ ID NO: 34 | | | |
| WS323-LZ | N183A | | SEQ ID NO: 22 | | | |
| WS331-LZ | P195A | | SEQ ID NO: 28 | | | |

TABLE 7-2
Expression and purity information of
LAG-3 D1-D2-LZ and mutants thereof

| Construct number | Mutation site | Expression (mg/L) | HPLC-SEC |
|---|---|---|---|
| W1161-LZ | Wild type | 26 | 68.58% |
| WS447-LZ | E201D | 55 | 81.67% |
| WS451-LZ | E201G | 40 | 80.27% |
| WS488-LZ | P207I | 67 | 79.67% |
| WS343-LZ | M212A | 72 | 78.27% |

### Example 9: Activity Assay of LAG-3 D1-D2-LZ Wild Type and Mutants Thereof

The binding capacity of the LAG-3 D1-D2-LZ wild type and the mutants thereof to human FGL1 was determined by ELISA using the same method as in Example 3. As shown in FIG. 10, compared with the W1161-LZ wild type, the binding capacity of mutants WS447-LZ and WS451-LZ to human FGL1 remained unchanged, and the binding capacity of mutants WS323-LZ and WS331-LZ to human FGL1 was improved. The change trend was consistent with that of the Fc-tagged proteins, suggesting that the effect produced by mutations is not influenced by the tag. Similarly, for the LAG-3 D1-D2-LZ fusion protein, other mutations in the D2 domain (such as N183A, G185A, Q186A, G187A, P190A, P195A, L199A, etc.) also increased the binding capacity to human FGL1 compared with the wild type.

The direct binding capacity of LAG-3 D1-D2-D3-D4-Fc wild type and mutants thereof to human MHCII was determined by FACS using Daudi cells as positive cells of human MHCII using the same method as in Example 3. As shown in FIG. 11, compared with the W1161-LZ wild type, the mutants WS447-LZ and WS451-LZ demonstrated improved direct binding capacity to human MHCII+ Daudi cells. The change trend was consistent with that of the Fc-tagged proteins, suggesting that the effect produced by mutations is not influenced by the tag. Similarly, for the LAG-3 D1-D2-LZ fusion protein, other mutations in the D2 domain (such as N183A, G185A, etc.) also increased the binding capacity to human MHCII compared with the wild type. Through the binding capacity to MHCII, FGL1 and the like, the mutants demonstrated significant in vitro and in vivo anti-tumor bioactivity.

### Example 10: Stability Test of LAG-3 D1-D2-LZ Wild Type and Mutants Thereof

The stability of the samples was evaluated using differential scanning calorimetry (DSC). Compared with the wild type W1161-LZ, the mutants WS447-LZ and WS451-LZ demonstrated improved Tm1 values, as shown in Table 8.

TABLE 8
DSC results of LAG-3 D1-D2-LZ wild type and mutants thereof

| Construct number | Mutation site | Tm1 | |
|---|---|---|---|
| W1161-LZ | Wild type | 55.95° | C. |
| WS447-LZ | E201D | 58.3° | C. |
| WS451-LZ | E201G | 57.97° | C. |

### Example 11: Preparation of LAG-3-Fab Wild Type and Mutants Thereof

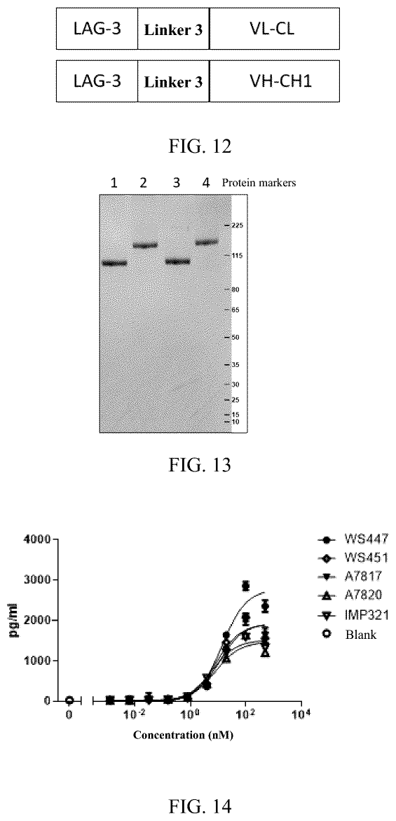

An illustrative schematic of the LAG-3-Fab is shown in FIG. 12. The specific structures of the wild type and the mutant constructs were LAG-3-Linker3-VL-CL and LAG-3-Linker3-VH-CH1. The LAG-3 in this example is LAG-3 D1-D2 or LAG-3 D1-1D2-1D3-1D4. The sequence information is shown in Table 9-1. The VL-CL and VH-CH1 can be paired to form an Fab fragment specific for the antigen, thereby allowing the fusion protein to form a dimeric structure. The Fab fragments formed by VL-CL and VH-CH1 in Y103-4A, 4A-D, 4A-G, Y103-7A, 7A-D, and 7A-G are specific for PD-L1; the Fab fragments formed by VL-CL and VH-CH1 in Y103-41B and Y103-71B are specific for PD-1. The expression and purification methods are the same as in Example 2. The multimer content was determined by high performance size exclusion chromatography (HPLC-SEC). The expression and purity of the mutants were improved as compared with the wild type, as shown in Table 9-2.

TABLE 9-1
Amino acid sequence information of LAG-3-Fab and mutants thereof

| Construct | Mutation | Sequence | | | | | |
|---|---|---|---|---|---|---|---|
| number | site | D1 | D2 | D3 | D4 | Linker3 | VL-CL/VH-CH1 |
| Y103-4A | Wild type | SEQ ID | SEQ ID | SEQ ID | SEQ ID | SEQ ID | SEQ ID NO: 4/ |
| | | NO: 10 | NO: 11 | NO: 12 | NO: 13 | NO: 9 | SEQ ID NO: 5 |
| 4A-D | E201D | | SEQ ID | SEQ ID | SEQ ID | | |
| | | | NO: 46 | NO: 12 | NO: 13 | | |
| 4A-G | E201G | | SEQ ID | SEQ ID | SEQ ID | | |
| | | | NO: 49 | NO: 12 | NO: 13 | | |
| Y103-7A | Wild type | | SEQ ID | NA | NA | | |
| | | | NO: 11 | | | | |
| 7A-D | E201D | | SEQ ID | NA | NA | | |
| | | | NO: 46 | | | | |
| 7A-G | E201G | | SEQ ID | NA | NA | | |
| | | | NO: 49 | | | | |
| Y103-4B | Wild type | | SEQ ID | SEQ ID | SEQ ID | | SEQ ID NO: 61/ |
| | | | NO: 11 | NO: 12 | NO: 13 | | SEQ ID NO: 62 |
| Y103-7B | Wild type | | SEQ ID | NA | NA | | |
| | | | NO: 11 | | | | |

TABLE 9-2
Expression and purity information
of LAG-3-Fab and mutants thereof

| Construct number | Mutation site | Expression (mg/L) | HPLC-SEC |
|---|---|---|---|
| Y103-4A | Wild type | 21 | 74.81% |
| 4A-D | E201D | 36 | 84.52% |
| 4A-G | E201G | 35 | 83.23% |
| Y103-7A | Wild type | 18 | 72.04% |
| 7A-D | E201D | 35 | 89.35% |
| 7A-G | E201G | 42 | 84.29% |

### Example 12: Activity Assay of LAG-3-Fab

The activity of the LAG-3 moiety in the LAG-3-Fab wild type and the mutants thereof, including the binding capacities to human FGL1 and human MHCII, were determined by ELISA and FACS using the method as in Example 3. The change trend induced by the mutations in the LAG3-Fab was basically the same as that in the LAG-3 D1-D2-Fc.

The activity of two moieties of the LAG-3-Fab, including the LAG-3 moiety and the anti-PD-L1 or anti-PD-1 Fab moiety, was detected by Biacore. An antigen was fixed on a CM5 chip by amino coupling, wherein the antigen coupling amount was 800 RU. The sample was diluted to an initial concentration using 1×HBS-EP+buffer, and then serially 2-fold diluted to four different concentrations. The diluted samples were loaded on a detection system in ascending order of concentration for detection, wherein the binding flow rate was 30 μL/min, the binding time was 120 s, and the dissociation time was 300 s. The chip was regenerated with a pH1.5Glycine solution, wherein the regeneration flow rate was L/min and the regeneration time was 30 s. After the detection was completed, data fitting was conducted on the resultant pattern by using the Biacore T200 Evaluation Software in a 11 binding fitting mode to obtain an equilibrium dissociation equilibrium constant (K_{D}).

The results are shown in Table 10. Y103-4A, Y103-7A, Y103-4B, and Y103-7B demonstrated strong binding capacity to hFGL1 antigen and PD-L1 (SB, Cat. No. 10084-H08H) or PD-1 (SB, Cat. No. HPLC-10377-H08H) antigen and higher affinity for hFGL1 as compared with IMP321. All of these exhibited significant in vitro and in vivo anti-tumor bioactivity.

TABLE 10
BIACORE assay of LAG-3-Fab

| | KD hFGL1 | KD PD-L1 or |
|---|---|---|
| Construct number | (M) | PD-1 (M) |
| IMP321 (LAG3 D1-D2-D3-D4-Fc) | 8.430E−09 | — |
| Y103-4A (LAG3 D1-D2-D3-D4-Fab) | 9.292E−10 | 2.234E−13 (PD-L1) |
| Y103-7A (LAG3 D1-D2-Fab) | 2.458E−09 | 3.648E−11 (PD-L1) |
| Y103-4B (LAG3 D1-D2-D3-D4-Fab) | 6.365E−10 | 1.071E−8 (PD-1) |
| Y103-7B (LAG3 D1-D2-Fab) | 3.257E−09 | 1.571E−8 (PD-1) |

### Example 13: Stability Test for LAG-3-Fab

The stability of the samples was evaluated using differential scanning calorimetry (DSC). Compared with IMP321, Y103-4A and Y103-4B demonstrated improved Tm1 values, and compared with W1161-WT, Y103-7A and Y103-7B demonstrated higher Tm1 values, as shown in Table 11.

TABLE 11
DSC results of LAG3-Fab

| Construct number | Tm1 | |
|---|---|---|
| IMP321 (LAG3 D1-D2-D3-D4-Fc) | 52.09° | C. |
| Y103-4A (LAG3 D1-D2-D3-D4-Fab) | 57.82° | C. |
| Y103-4B (LAG3 D1-D2-D3-D4-Fab) | 57.22° | C. |
| W1161-WT (LAG3 D1-D2-Fc) | 56.4° | C. |
| Y103-7A (LAG3 D1-D2-Fab) | 58.40° | C. |
| Y103-7B (LAG3 D1-D2-Fab) | 57.86° | C. |

The samples were diluted to 0.5 mg/mL, added into 1.5-mL EP tubes at 100 μL/tube, and incubated in a water bath at 40° C. for thermal acceleration for 14 days. The start of the test was designated as D0, and the 14^{th} day was designated as D14. After two weeks, the D0 and D14 samples were subjected to SDS-PAGE. As shown in FIG. 13, after 14 days of thermal acceleration, no change was found in LAG-3-Fab (including LAG-3 D1-D2-Fab and LAG-3 D1-D2-D3-D4-Fab constructs); the IMP321 (LAG-3 D1-D2-D3-D4-Fc construct) was completely degraded at 14 days of thermal acceleration (see FIG. 8), and impurity bands were found in W1161-WT (LAG-3 D1-D2-Fc construct) after 14 days of thermal acceleration (see FIG. 5), indicating that the stability of the LAG3-Fab is significantly superior to that of the LAG-3-Fc.

### Example 14

LAG-3 D1-D2-D3-Fc and mutants thereof were constructed, and the specific structure was D1-D2-D3-Linker-Fc. The mutation sites of the mutants were E201D, E201G, P207I, and M212A, and the sequences of the linker and Fc are respectively set forth in SEQ ID NO: 6 and SEQ ID NO: 1. Similar effects were also achieved by repeating the experimental procedures of Examples 2-4 above, i.e., the expression and purity of the mutants were significantly improved as compared to the wild type (>50% for expression and >15% for purity), and the affinity and stability were also improved. Through the binding capacity to MHCII, FGL1 and the like, the mutants demonstrated significant in vitro and in vivo anti-tumor bioactivity.

### Example 15

The point mutations validated in the above examples were combined to give fusion protein constructs LAG-3 D1-D2-Fc, LAG-3 D1-D2-D3-D4-Fc, LAG-3 D1-D2-LZ, and LAG-3-Fab. The specific combinations of mutations are shown in Table 12 below. The specific sequences can be found in Examples 1-12 above, and the sequences of VL-CL and VH-CH1 in the LAG-3-Fab are shown in SEQ ID NO: 4 and SEQ ID NO: 5, respectively. The structures of LAG-3 D1-D2-Fc and LAG-3 D1-D2-D3-D4-Fc are shown in FIG. 1, the structure of LAG-3 D1-D2-LZ is shown in FIG. 9, and the structure of LAG-3 Fab is shown in FIG. 12. The experimental procedures of examples 2-4 above were repeated. The results suggest that the combinations of two point mutations, three point mutations, and four point mutations also achieved similar effects, such as one or more of the following: high expression, high purity, high stability, and high affinity. Moreover, the results of the combinations of two point mutations, three point mutations, and four point mutations are all superior to those of the single point mutations. Through the binding capacity to MHCII, FGL1 and the like, the mutants demonstrated significant in vitro and in vivo anti-tumor bioactivity.

TABLE 12
Combination of mutation sites

| Combination of two | Combination of three | Combination of four |
|---|---|---|
| point mutations | point mutations | point mutations |
| P207E, M212A | P207E, M212A, E201D | P207E, M212A, E201D, H197A |
| P207E, E201D | P207I, M212A, E201G | P207E, N183A, E201D, H197A |
| P207I, E201G | P207I, H197A, E201D | P207I, M212A, E201D, N183A |
| P207E, N183A | P207E, H197A, E201G | P207E, E201D, P195A, H197A |
| H197A, E201D | P207E, N183A, E201D | M212A, E201D, N183A, H197A |
| H197A, E201G | P207E, Q186A, E201G | P207E, M212A, E201G, H197A |
| E201D, N183A | P207I, E201D, Q186A | P207E, N183A, E201G, H197A |
| E201G, Q186A | P207R, E201G, P195A | P207E, M212A, E201G, N183A |
| E201D, G185A | P207D, E201D, G187A | P207E, E201G, G185A, H197A |
| E201D, Q186A | E201D, H197A, Q186A | M212A, E201G, Q186A, H197A |
| E201D, G187A | E201D, P190A, N183A | P207E, M212A, S177A, Q186A |
| E201G, P190A | M212A, E201G, G187A | E201D, H197A, Q186A, H198Y |
| E201G, P195A | E201G, H197A, L199A | E201G, G187A, P195A, L199A |
| E201G, L199A | E201D, Q186A, P195A | E201D, Q186A, G187A, P190A |

### Example 16: Bioactivity Assay for Inducing Cytokine Release in Monocytes

JAWSII cells in the logarithmic growth phase (from ATCC® CRL-11904™) were seeded on a 96-well plate at 5×10⁴ cells/100 μL/well. The sample was diluted to 500 nM with a buffer and serially 5-fold diluted to nine different concentrations. The diluted samples were then added to a 96-well plate at 100 μL/well. The 96-well plate was incubated in an incubator at 5% CO₂/37° C. for 48 h, and centrifuged at 300×g for 5 min. The cell supernatant was collected, and the expression of mTNF-α in the cell supernatant was detected with an mTNF-α ELISA kit (R&D, DY410-05).

The results are shown in FIG. 14. The mutants WS447, WS451, A7817, and A7820 were all capable of inducing mTNF-α release in immature mouse dendritic cells JAWSII with comparable or superior activity as compared to IMP321. This suggests that constructs WS447, WS451, A7817, and A7820 can induce positive signaling of MHCII molecules and promote the secretion of pro-inflammatory factors and chemokines in antigen-presenting cells (APCs). Activated APCs can enhance existing immune responses.

SEQUENCE LISTING

| | Sequence | | SEQ ID |
|---|---|---|---|
| Classification | code | Amino acid sequence | NO. |
| Fc | Fc | APELLGGPSVFLFPPKPKDTLMISRTPEVTCVVVDVSHEDPE | 1 |
| | | VKFNWYVDGVEVHNAKTKPREEQYNSTYRVVSVLTVLHQD | |
| | | WLNGKEYKCKVSNKALPAPIEKTISKAKGQPREPQVYTLPPS | |
| | | RDELTKNQVSLTCLVKGFYPSDIAVEWESNGQPENNYKTTPP | |
| | | VLDSDGSFFLYSKLTVDKSRWQQGNVFSCSVMHEALHNHY | |
| | | TQKSLSLSPGK | |
| Leucine zipper | c-JUN-His | RIARLEEKVKTLKAQNSELASTANMLREQVAQLKQKVMNH | 2 |
| | | HHHHH | |
| | c-FOS-His | LTDTLQAETDQLEDEKSALQTEIANLLKEKEKLEFILAAHHH | 3 |
| | | HHH | |
| Anti-PD-L1 Fab | VL-CL | DIQMTQSPSSLSASVGDRVTITCRASQDVSTAVAWYQQKPGK | 4 |
| | | APKLLIYSASFLYSGVPSRFSGSGSGTDFTLTISSLQPEDFATY | |
| | | YCQQYLYHPATFGQGTKVEIKRTVAAPSVFIFPPSDEQLKSGT | |
| | | ASVVCLLNNFYPREAKVQWKVDNALQSGNSQESVTEQDSK | |
| | | DSTYSLSSTLTLSKADYEKHKVYACEVTHQGLSSPVTKSFNR | |
| | | GEC | |
| | VH-CH1 | EVQLVESGGGLVQPGGSLRLSCAASGFTFSDSWIHWVRQAP | 5 |
| | | GKGLEWVAWISPYGGSTYYADSVKGRFTISADTSKNTAYLQ | |
| | | MNSLRAEDTAVYYCARRHWPGGFDYWGQGTLVTVSSASTK | |
| | | GPSVFPLAPSSKSTSGGTAALGCLVKDYFPEPVTVSWNSGAL | |
| | | TSGVHTFPAVLQSSGLYSLSSVVTVPSSSLGTQTYICNVNHKP | |
| | | SNTKVDKKVEPKSC | |
| Linker peptide | Linker | GGGGSGGGGSEPKSCDKTHTCPPCP | 6 |
| | Linker1 | DDDDKGSGSGEPKSCDKTHTCPPCP | 7 |
| | Linker2 | GGGGSGGGGSEPKSSDKTHTCPPCP | 8 |
| | Linker3 | GGGGSGGGGSGGGGS | 9 |
| LAG-3 domains | D1 | LQPGAEVPVVWAQEGAPAQLPCSPTIPLQDLSLLRRAGVTW | 10 |
| (wild type) | | QHQPDSGPPAAAPGHPLAPGPHPAAPSSWGPRPRRYTVLSVG | |
| | | PGGLRSGRLPLQPRVQLDERGRQRGDFSLWLRPARRADAGE | |
| | | YRAAVHLRDRALSCRLRLRLGQASM | |
| | D2 | TASPPGSLRASDWVILNCSFSRPDRPASVHWFRNRGQGRVPV | 11 |
| | | RESPHHHLAESFLFLPQVSPMDSGPWGCILTYRDGFNVSIMY | |
| | | NLTVLG | |
| | D3 | LEPPTPLTVYAGAGSRVGLPCRLPAGVGTRSFLTAKWTPPGG | 12 |
| | | GPDLLVTGDNGDFTLRLEDVSQAQAGTYTCHIHLQEQQLNA | |
| | | TVTLAIIT | |
| | D4 | VTPKSFGSPGSLGKLLCEVTPVSGQERFVWSSLDTPSQRSFS | 13 |
| | | GPWLEAQEAQLLSQPWQCQLYQGERLLGAAVYFTELSSPG | |
| LAG-3 D2 | D2-R188A | TASPPGSLRASDWVILNCSFSRPDRPASVHWFRNRGQGAVP | 14 |
| domain (mutant) | | VRESPHHHLAESFLFLPQVSPMDSGPWGCILTYRDGFNVSIM | |
| | | YNLTVLG | |
| | D2-R192A | TASPPGSLRASDWVILNCSFSRPDRPASVHWFRNRGQGRVPV | 15 |
| | | AESPHHHLAESFLFLPQVSPMDSGPWGCILTYRDGFNVSIMY | |
| | | NLTVLG | |
| | D2-H196A | TASPPGSLRASDWVILNCSFSRPDRPASVHWFRNRGQGRVPV | 16 |
| | | RESPAHHLAESFLFLPQVSPMDSGPWGCILTYRDGFNVSIMY | |
| | | NLTVLG | |
| | D2-H197A | TASPPGSLRASDWVILNCSFSRPDRPASVHWFRNRGQGRVPV | 17 |
| | | RESPHAHLAESFLFLPQVSPMDSGPWGCILTYRDGFNVSIMY | |
| | | NLTVLG | |
| | D2-P172A | TASPPGSLRASDWVILNCSFSRADRPASVHWFRNRGQGRVP | 18 |
| | | VRESPHHHLAESFLFLPQVSPMDSGPWGCILTYRDGFNVSIM | |
| | | YNLTVLG | |
| | D2-P175A | TASPPGSLRASDWVILNCSFSRPDRAASVHWFRNRGQGRVP | 19 |
| | | VRESPHHHLAESFLFLPQVSPMDSGPWGCILTYRDGFNVSIM | |
| | | YNLTVLG | |
| | D2-S177A | TASPPGSLRASDWVILNCSFSRPDRPAAVHWFRNRGQGRVP | 20 |
| | | VRESPHHHLAESFLFLPQVSPMDSGPWGCILTYRDGFNVSIM | |
| | | YNLTVLG | |
| | D2-V178A | TASPPGSLRASDWVILNCSFSRPDRPASAHWFRNRGQGRVPV | 21 |
| | | RESPHHHLAESFLFLPQVSPMDSGPWGCILTYRDGFNVSIMY | |
| | | NLTVLG | |
| | D2-N183A | TASPPGSLRASDWVILNCSFSRPDRPASVHWFRARGQGRVPV | 22 |
| | | RESPHHHLAESFLFLPQVSPMDSGPWGCILTYRDGFNVSIMY | |
| | | NLTVLG | |
| LAG-3 D2 | D2-G185A | TASPPGSLRASDWVILNCSFSRPDRPASVHWFRNRAQGRVPV | 23 |
| domain (mutant) | | RESPHHHLAESFLFLPQVSPMDSGPWGCILTYRDGFNVSIMY | |
| | | NLTVLG | |
| | D2-Q186A | TASPPGSLRASDWVILNCSFSRPDRPASVHWFRNRGAGRVPV | 24 |
| | | RESPHHHLAESFLFLPQVSPMDSGPWGCILTYRDGFNVSIMY | |
| | | NLTVLG | |
| | D2-G187A | TASPPGSLRASDWVILNCSFSRPDRPASVHWFRNRGQARVPV | 25 |
| | | RESPHHHLAESFLFLPQVSPMDSGPWGCILTYRDGFNVSIMY | |
| | | NLTVLG | |
| | D2-V189A | TASPPGSLRASDWVILNCSFSRPDRPASVHWFRNRGQGRAPV | 26 |
| | | RESPHHHLAESFLFLPQVSPMDSGPWGCILTYRDGFNVSIMY | |
| | | NLTVLG | |
| | D2-P190A | TASPPGSLRASDWVILNCSFSRPDRPASVHWFRNRGQGRVA | 27 |
| | | VRESPHHHLAESFLFLPQVSPMDSGPWGCILTYRDGFNVSIM | |
| | | YNLTVLG | |
| | D2-P195A | TASPPGSLRASDWVILNCSFSRPDRPASVHWFRNRGQGRVPV | 28 |
| | | RESAHHHLAESFLFLPQVSPMDSGPWGCILTYRDGFNVSIMY | |
| | | NLTVLG | |
| | D2-L199A | TASPPGSLRASDWVILNCSFSRPDRPASVHWFRNRGQGRVPV | 29 |
| | | RESPHHHAAESFLFLPQVSPMDSGPWGCILTYRDGFNVSIMY | |
| | | NLTVLG | |
| | D2-F203A | TASPPGSLRASDWVILNCSFSRPDRPASVHWFRNRGQGRVPV | 30 |
| | | RESPHHHLAESALFLPQVSPMDSGPWGCILTYRDGFNVSIMY | |
| | | NLTVLG | |
| | D2-Q208A | TASPPGSLRASDWVILNCSFSRPDRPASVHWFRNRGQGRVPV | 31 |
| | | RESPHHHLAESFLFLPAVSPMDSGPWGCILTYRDGFNVSIMY | |
| | | NLTVLG | |
| | D2-S210A | TASPPGSLRASDWVILNCSFSRPDRPASVHWFRNRGQGRVPV | 32 |
| | | RESPHHHLAESFLFLPQVAPMDSGPWGCILTYRDGFNVSIMY | |
| | | NLTVLG | |
| | D2-P211A | TASPPGSLRASDWVILNCSFSRPDRPASVHWFRNRGQGRVPV | 33 |
| | | RESPHHHLAESFLFLPQVSAMDSGPWGCILTYRDGFNVSIMY | |
| | | NLTVLG | |
| LAG-3 D2 | D2-M212A | TASPPGSLRASDWVILNCSFSRPDRPASVHWFRNRGQGRVPV | 34 |
| domain (mutant) | | RESPHHHLAESFLFLPQVSPADSGPWGCILTYRDGFNVSIMY | |
| | | NLTVLG | |
| | D2-S214A | TASPPGSLRASDWVILNCSFSRPDRPASVHWFRNRGQGRVPV | 35 |
| | | RESPHHHLAESFLFLPQVSPMDAGPWGCILTYRDGFNVSIMY | |
| | | NLTVLG | |
| | D2-P216A | TASPPGSLRASDWVILNCSFSRPDRPASVHWFRNRGQGRVPV | 36 |
| | | RESPHHHLAESFLFLPQVSPMDSGAWGCILTYRDGFNVSIMY | |
| | | NLTVLG | |
| | D2-G218A | TASPPGSLRASDWVILNCSFSRPDRPASVHWFRNRGQGRVPV | 37 |
| | | RESPHHHLAESFLFLPQVSPMDSGPWACILTYRDGFNVSIMY | |
| | | NLTVLG | |
| | D2-H198G | TASPPGSLRASDWVILNCSFSRPDRPASVHWFRNRGQGRVPV | 38 |
| | | RESPHHGLAESFLFLPQVSPMDSGPWGCILTYRDGFNVSIMY | |
| | | NLTVLG | |
| | D2-H198L | TASPPGSLRASDWVILNCSFSRPDRPASVHWFRNRGQGRVPV | 39 |
| | | RESPHHLLAESFLFLPQVSPMDSGPWGCILTYRDGFNVSIMY | |
| | | NLTVLG | |
| | D2-H198M | TASPPGSLRASDWVILNCSFSRPDRPASVHWFRNRGQGRVPV | 40 |
| | | RESPHHMLAESFLFLPQVSPMDSGPWGCILTYRDGFNVSIMY | |
| | | NLTVLG | |
| | D2-H198W | TASPPGSLRASDWVILNCSFSRPDRPASVHWFRNRGQGRVPV | 41 |
| | | RESPHHWLAESFLFLPQVSPMDSGPWGCILTYRDGFNVSIMY | |
| | | NLTVLG | |
| | D2-H198Y | TASPPGSLRASDWVILNCSFSRPDRPASVHWFRNRGQGRVPV | 42 |
| | | RESPHHYLAESFLFLPQVSPMDSGPWGCILTYRDGFNVSIMY | |
| | | NLTVLG | |
| | D2-H198V | TASPPGSLRASDWVILNCSFSRPDRPASVHWFRNRGQGRVPV | 43 |
| | | RESPHHVLAESFLFLPQVSPMDSGPWGCILTYRDGFNVSIMY | |
| | | NLTVLG | |
| | D2-E201R | TASPPGSLRASDWVILNCSFSRPDRPASVHWFRNRGQGRVPV | 44 |
| | | RESPHHHLARSFLFLPQVSPMDSGPWGCILTYRDGFNVSIMY | |
| | | NLTVLG | |
| LAG-3 D2 | D2-E201N | TASPPGSLRASDWVILNCSFSRPDRPASVHWFRNRGQGRVPV | 45 |
| domain (mutant) | | RESPHHHLANSFLFLPQVSPMDSGPWGCILTYRDGFNVSIMY | |
| | | NLTVLG | |
| | D2-E201D | TASPPGSLRASDWVILNCSFSRPDRPASVHWFRNRGQGRVPV | 46 |
| | | RESPHHHLADSFLFLPQVSPMDSGPWGCILTYRDGFNVSIMY | |
| | | NLTVLG | |
| | D2-E201Q | TASPPGSLRASDWVILNCSFSRPDRPASVHWFRNRGQGRVPV | 47 |
| | | RESPHHHLAQSFLFLPQVSPMDSGPWGCILTYRDGFNVSIMY | |
| | | NLTVLG | |
| | D2-E201H | TASPPGSLRASDWVILNCSFSRPDRPASVHWFRNRGQGRVPV | 48 |
| | | RESPHHHLAHSFLFLPQVSPMDSGPWGCILTYRDGFNVSIMY | |
| | | NLTVLG | |
| | D2-E201G | TASPPGSLRASDWVILNCSFSRPDRPASVHWFRNRGQGRVPV | 49 |
| | | RESPHHHLAGSFLFLPQVSPMDSGPWGCILTYRDGFNVSIMY | |
| | | NLTVLG | |
| | D2-E201F | TASPPGSLRASDWVILNCSFSRPDRPASVHWFRNRGQGRVPV | 50 |
| | | RESPHHHLAFSFLFLPQVSPMDSGPWGCILTYRDGFNVSIMY | |
| | | NLTVLG | |
| | D2-E201S | TASPPGSLRASDWVILNCSFSRPDRPASVHWFRNRGQGRVPV | 51 |
| | | RESPHHHLASSFLFLPQVSPMDSGPWGCILTYRDGFNVSIMY | |
| | | NLTVLG | |
| | D2-P207R | TASPPGSLRASDWVILNCSFSRPDRPASVHWFRNRGQGRVPV | 52 |
| | | RESPHHHLAESFLFLRQVSPMDSGPWGCILTYRDGFNVSIMY | |
| | | NLTVLG | |
| | D2-P207D | TASPPGSLRASDWVILNCSFSRPDRPASVHWFRNRGQGRVPV | 53 |
| | | RESPHHHLAESFLFLDQVSPMDSGPWGCILTYRDGFNVSIMY | |
| | | NLTVLG | |
| | D2-P207E | TASPPGSLRASDWVILNCSFSRPDRPASVHWFRNRGQGRVPV | 54 |
| | | RESPHHHLAESFLFLEQVSPMDSGPWGCILTYRDGFNVSIMY | |
| | | NLTVLG | |
| | D2-P207I | TASPPGSLRASDWVILNCSFSRPDRPASVHWFRNRGQGRVPV | 55 |
| | | RESPHHHLAESFLFLIQVSPMDSGPWGCILTYRDGFNVSIMY | |
| | | NLTVLG | |
| LAG-3 D2 | D2-P207M | TASPPGSLRASDWVILNCSFSRPDRPASVHWFRNRGQGRVPV | 56 |
| domain (mutant) | | RESPHHHLAESFLFLMQVSPMDSGPWGCILTYRDGFNVSIM | |
| | | YNLTVLG | |
| | D2-P207S | TASPPGSLRASDWVILNCSFSRPDRPASVHWFRNRGQGRVPV | 57 |
| | | RESPHHHLAESFLFLSQVSPMDSGPWGCILTYRDGFNVSIMY | |
| | | NLTVLG | |
| | D2-P207T | TASPPGSLRASDWVILNCSFSRPDRPASVHWFRNRGQGRVPV | 58 |
| | | RESPHHHLAESFLFLTQVSPMDSGPWGCILTYRDGFNVSIMY | |
| | | NLTVLG | |
| | D2-P207Y | TASPPGSLRASDWVILNCSFSRPDRPASVHWFRNRGQGRVPV | 59 |
| | | RESPHHHLAESFLFLYQVSPMDSGPWGCILTYRDGFNVSIMY | |
| | | NLTVLG | |
| | D2-V209T | TASPPGSLRASDWVILNCSFSRPDRPASVHWFRNRGQGRVPV | 60 |
| | | RESPHHHLAESFLFLPQTSPMDSGPWGCILTYRDGFNVSIMY | |
| | | NLTVLG | |
| Anti-PD-1 Fab | VL-CL | EIVLTQSPATLSLSPGERATLSCRASKGVSTSGYSYLHWYQQ | 61 |
| | | KPGQAPRLLIYLASYLESGVPARFSGSGSGTDFTLTIS | |
| | | SLEPEDFAVYYCQHSRDLPLTFGGGTKVEIKRTVAAPSVFIFPP | |
| | | SDEQLKSGTASVVCLLNNFYPREAKVQWKVDNALQSGNSQ | |
| | | ESVTEQDSKDSTYSLSSTLTLSKADYEKHKVYACEVTHQGLS | |
| | | SPVTKSFNRGEC | |
| | VH-CH1 | QVQLVQSGVEVKKPGASVKVSCKASGYTFTNYYMYWVRQ | 62 |
| | | APGQGLEWMGGINPSNGGTNFNEKFKNRVTLTTDSSTTTAY | |
| | | MELKSLQFDDTAVYYCARRDYRFDMGFDYWGQGTTVTVSS | |
| | | ASTKGPSVFPLAPSSKSTSGGTAALGCLVKDYFPEPVTVSWN | |
| | | SGALTSGVHTFPAVLQSSGLYSLSSVVTVPSSSLGTQTYICNV | |
| | | NHKPSNTKVDKKVEPKSC | |
| LAG-3 D1-D2 | D1-D2 | LQPGAEVPVVWAQEGAPAQLPCSPTIPLQDLSLLRRAGVTW | 63 |
| domains (wild | | QHQPDSGPPAAAPGHPLAPGPHPAAPSSWGPRPRRYTVLSVG | |
| type) | | PGGLRSGRLPLQPRVQLDERGRQRGDFSLWLRPARRADAGE | |
| | | YRAAVHLRDRALSCRLRLRLGQASMTASPPGSLRASDWVIL | |
| | | NCSFSRPDRPASVHWFRNRGQGRVPVRESPHHHLAESFLFLP | |
| | | QVSPMDSGPWGCILTYRDGFNVSIMYNLTVLG | |
| Uniprot P18627 | | MWEAQFLGLLFLQPLWVAPVKPLQPGAEVPVVWAQEGAPA | 64 |
| human LAG-3 | | QLPCSPTIPLQDLSLLRRAGVTWQHQPDSGPPAAAPGHPLAP | |
| sequence | | GPHPAAPSSWGPRPRRYTVLSVGPGGLRSGRLPLQPRVQLDE | |
| (including signal | | RGRQRGDFSLWLRPARRADAGEYRAAVHLRDRALSCRLRL | |
| peptide) | | RLGQASMTASPPGSLRASDWVILNCSFSRPDRPASVHWFRN | |
| | | RGQGRVPVRESPHHHLAESFLFLPQVSPMDSGPWGCILTYRD | |
| | | GFNVSIMYNLTVLGLEPPTPLTVYAGAGSRVGLPCRLPAGVG | |
| | | TRSFLTAKWTPPGGGPDLLVTGDNGDFTLRLEDVSQAQAGT | |
| | | YTCHIHLQEQQLNATVTLAIITVTPKSFGSPGSLGKLLCEVTP | |
| | | VSGQERFVWSSLDTPSQRSFSGPWLEAQEAQLLSQPWQCQL | |
| | | YQGERLLGAAVYFTELSSPGAQRSGRAPGALPAGHLLLFLIL | |
| | | GVLSLLLLVTGAFGFHLWRRQWRPRRFSALEQGIHPPQAQS | |
| | | KIEELEQEPEPEPEPEPEPEPEPEPEQL | |

1. An LAG-3 protein mutant, comprising mutations at one or more of the following positions on the basis of the domain 2 of the LAG-3 protein: 201, 188, 192, 196, 197, 172, 175, 177, 178, 183, 185, 186, 187, 189, 190, 195, 199, 203, 208, 210, 211, 212, 214, 216, 218, 198, 207 and 209, preferably, mutations at one or more of the following positions on the basis of the domain 2 of the LAG-3 protein: 201, 177, 183, 185, 186, 187, 190, 195, 197, 198, 199, 207, 212, 214 and 218, or preferably, mutations at one or more of the following positions on the basis of the domain 2 of the LAG-3 protein: 201, 183, 185, 186, 187, 190, 195, 197, 199, 207 and 212, wherein the numbering of the amino acid positions corresponds to the numbering of the sequence set forth in SEQ ID NO: 63, and preferably, the sequence of the domain 2 of the LAG-3 protein is set forth in SEQ ID NO: 11;
preferably, the LAG-3 protein comprises the domain 1 and the domain 2, and optionally the domain 3 and/or the domain 4;
preferably, the LAG-3 protein comprises the intact LAG-3 protein or an LAG-3 protein fragment, wherein the LAG-3 protein fragment is selected from the group consisting of:
(1) an LAG-3 protein fragment comprising or consisting of domain 1 and domain 2;
(2) an LAG-3 protein fragment comprising or consisting of domain 1, domain 2, and domain 3; or
(3) an LAG-3 protein fragment comprising or consisting of domain 1, domain 2, domain 3, and domain 4.

2. The LAG-3 protein mutant according to claim 1, comprising one or more of the following mutations on the basis of the domain 2 of the LAG-3 protein: E201R, E201N, E201D, E201Q, E201H, E201G, E201F, E201S, R188A, R192A, H196A, H197A, P172A, P175A, S177A, V178A, N183A, G185A, Q186A, G187A, V189A, P190A, P195A, L199A, F203A, Q208A, S210A, P211A, M212A, S214A, P216A, G218A, H198G, H198L, H198M, H198W, H198Y, H198V, P207R, P207D, P207E, P207I, P207M, P207S, P207T, P207Y and V209T, preferably, E201D, E201G, P207E, P207I, P207R, P207D, M212A, P207M, S177A, P207T, Q186A, G187A, H197A, H198Y, G185A, L199A, N183A, P190A, P195A, S214A, P207Y, G218A, H198W and H198V, or preferably, E201D, E201G, N183A, G185A, Q186A, G187A, P190A, P195A, H197A, L199A, P207E, P207I, P207R, P207D, M212A and P207M.

3. The LAG-3 protein mutant according to claim 1, wherein the mutation present in the domain 2 of the LAG-3 protein is selected from the group consisting of: E201R; E201N; E201D; E201Q; E201H; E201G; E201F; E201S; P207E and E201D; P207I and E201G; E201D and Q186A; H197A and E201G; P207I, E201D and Q186A; E201D, Q186A and P195A; P207E, Q186A and E201G; P207E, E201D, P195A and H197A; P207I, M212A, E201D and N183A; and P207E, M212A, E201G and N183A, N183A, G185A, Q186A, G187A, P190A, P195A, L199A and E201D: R188A; R192A; H196A; H197A; P172A; P175A; S177A; V178A; N183A; G185A; Q186A; G187A; V189A; P190A; P195A; L199A; F203A; Q208A; S210A; P211A; M212A; S214A; P216A; G218A; H198G; H198L; H198M; H198W; H198Y; H198V; P207R; P207D; P207E; P207I; P207M; P207S; P207T; P207Y; V209T; P207E and M212A; or preferably, the mutation is selected from the group consisting of: E201D: E201G: P207E and E201D: P207I and E201G: E201D and Q186A: H197A and E201G; and P207I, E201D and Q186A: N183A; G185A; Q186A; G187A; P190A; P195A; L199A; P207E and M212A; preferably, the domain 2 of the LAG-3 protein mutant comprises a sequence set forth in any one of SEQ ID NOs: 14-60.

4. An LAG-3 fusion protein, comprising a structure as follows: a structural unit 1—a structural unit 2, wherein the structural unit 1 is selected from LAG-3 D1-D2, LAG-3 D1-D2-D3, or LAG-3 D1-D2-D3-D4, wherein D1 denotes the domain 1 of the LAG-3, D2 denotes the domain 2 of the LAG-3 protein or a domain 2 mutant, D3 denotes the domain 3 of the LAG-3, and D4 denotes the domain 4 of the LAG-3;
preferably, D1 has a sequence set forth in SEQ ID NO: 10 or set forth in amino acids 37-167 of SEQ ID NO: 64;
D2 has a sequence set forth in SEQ ID NO: 11, set forth in the sequence of the domain 2 of the LAG-3 protein mutant according to claim 1, or set forth in amino acids 168-252 of SEQ ID NO: 64;
D3 has a sequence set forth in SEQ ID NO: 12 or set forth in amino acids 265-343 of SEQ ID NO: 64;
D4 has a sequence set forth in SEQ ID NO: 13 or set forth in amino acids 348-419 of SEQ ID NO: 64;
the structural unit 2 is a structural unit enabling the LAG-3 fusion protein to form a dimer or multimer, and is preferably selected from: an Fc fragment (preferably the Fc region is an Fc region from an IgG (e.g., IgG1, IgG2, IgG3 or IgG4) antibody, preferably having a sequence set forth in SEQ ID NO: 1); a VL-CL or VH-CH1 of an Fab fragment, wherein the VL-CL and VH-CH1 are paired to form an Fab fragment or Fab′ fragment specific for an antigen (preferably when the VL-CL has a sequence set forth in SEQ ID NO: 4, the VH-CH1 has a sequence set forth in SEQ ID NO: 5; or when the VL-CL has a sequence set forth in SEQ ID NO: 61, the VH-CH1 has a sequence set forth in SEQ ID NO: 62); or a c-JUN (preferably having a sequence set forth in positions 1-39 of SEQ ID NO: 2) or c-FOS (preferably having a sequence set forth in positions 1-39 of SEQ ID NO: 3), wherein the c-JUN and c-FOS are paired to form a leucine zipper; when D2 denotes the native D2 domain of the LAG-3, the structural unit 2 is either VL-CL or VH-CH1 of the Fab fragment.

5. An LAG-3 fusion protein dimer or multimer, comprising the LAG-3 fusion protein according to claim 4, wherein the structural unit 1 in the LAG-3 fusion protein dimer or multimer is identical or different.

6. The LAG-3 fusion protein dimer or multimer according to claim 5, wherein the LAG-3 fusion protein dimer or multimer is an LAG-3 fusion protein dimer, and the structural unit 1 is selected from LAG-3 D1-D2, LAG-3 D1-D2-D3, or LAG-3 D1-D2-D3-D4,
wherein D1 denotes the domain 1 of the LAG-3, D2 denotes the domain 2 of the LAG-3 protein or a domain 2 mutant, D3 denotes the domain 3 of the LAG-3, and D4 denotes the domain 4 of the LAG-3;
preferably, D1 has a sequence set forth in SEQ ID NO: 10 or set forth in amino acids 37-167 of SEQ ID NO: 64;
D2 has a sequence set forth in SEQ ID NO: 11, set forth in the sequence of the domain 2 of the LAG-3 protein mutant according to claim 1, or set forth in amino acids 168-252 of SEQ ID NO: 64;
D3 has a sequence set forth in SEQ ID NO: 12 or set forth in amino acids 265-343 of SEQ ID NO: 64;
D4 has a sequence set forth in SEQ ID NO: 13 or set forth in amino acids 348-419 of SEQ ID NO: 64;
the structural unit 2 is selected from:
(1) an Fc fragment, wherein preferably, the Fc fragment has a sequence set forth in SEQ ID NO: 1; or
(2) a VL-CL or VH-CH1, wherein the VL-CL and VH-CH1, as two structural units 2 in the LAG-3 fusion protein dimer, are paired to form an Fab fragment specific for an antigen; preferably the antigen is selected from a tumor cell surface antigen, an immune cell surface antigen, a virus, a bacterium, an endotoxin, or a cytokine, e.g., CD3, SLAMF7, CD38, BCMA, CD16a, CEA, PD-L1, PD-1, CTLA-4, TIGIT, LAG-3, VEGF, B7-H3, TGF-β or IL-10; preferably, the VL-CL of the Fab fragment has a sequence set forth in SEQ ID NO: 4, and the VH-CH1 has a sequence set forth in SEQ ID NO: 5.

7. The LAG-3 fusion protein according to claim 4, wherein the LAG-3 D1, D2, D3, D4, and the structural units are connected with or without a linker, and preferably the linker is selected from a sequence set forth in any one of SEQ ID NOs: 6-9.

8. A conjugate, comprising the LAG-3 protein mutant according to claim 1 and a conjugated moiety, wherein the conjugated moiety is a purification tag (e.g., His-tag, Fc-tag), a detectable label, a drug, a prodrug, a toxin, a cytokine, protein (e.g., an enzyme), a virus, a lipid, a biological response modulator (e.g., an immunomodulator), PEG, a hormone, a polypeptide, an oligonucleotide, a diagnostic agent, a cytotoxic agent, or a combination thereof; preferably, the conjugated moiety is a radioisotope, a fluorescent substance, a chemiluminescent substance, a colored substance, a chemotherapeutic agent, a biotoxin, polyethylene glycol, or an enzyme.

9. A pharmaceutical composition, comprising the LAG-3 protein mutant according to claim 1, wherein
preferably, the pharmaceutical composition further comprises at least one drug for treating a cancer or an infectious disease; preferably the drug is selected from a chemotherapeutic drug, an immunotherapeutic drug, or a combination thereof; preferably, the drug is selected from a radiotherapeutic agent, a chemotherapeutic agent (e.g., paclitaxels, anthracyclines, gemcitabine), a therapeutic antibody (e.g., rituximab, cetuximab, edrecolomab, trastuzumab, an anti-PD-1 antibody, an anti-PD-L1 antibody), a cytokine, a polypeptide, an antimetabolite, or a combination thereof;
preferably, the pharmaceutical composition further comprises at least one immune checkpoint regulator selected from: (a) an antagonist of an inhibitory immune checkpoint molecule; and (b) an agonist of a stimulatory immune checkpoint molecule.

10. A method for modulating an immune response or immunostimulation or treating or diagnosing a cancer or Parkinson's disease, or in preparing a medicament, an immunostimulant or an adjuvant for modulating an immune response or treating or diagnosing a cancer or Parkinson's disease, wherein the LAG-3 protein mutant according to claim 1 is administered to a subject in need thereof.
